(19) United States
(12) Patent Application Publication
(10) Pub. No.: US 2023/0302156 A1
(43) Pub. Date: Sep. 28, 2023

(54) REDOX-RESPONSIVE NANOPARTICLE COMPOSITIONS FOR OCULAR DELIVERY OF THERAPEUTICS

(71) Applicant: OHIO STATE INNOVATION FOUNDATION, Columbus, OH (US)

(72) Inventors: Pengfei JIANG, New York, NY (US); Katelyn Elizabeth REILLY, Columbus, OH (US)

(21) Appl. No.: 18/011,399

(22) PCT Filed: Jun. 22, 2021
(86) PCT No.: PCT/US2021/038369

§ 371 Date: Dec. 19, 2022

Related U.S. Application Data
(60) Provisional application No. 63/055,573, filed on Jul. 23, 2020.
(60) Provisional application No. 63/042,086, filed on Jun. 22, 2020.

(51) Int. Cl. A61K 47/69; A61K 9/00; A61P 27/02

(52) U.S. Cl. CPC A61K 47/6933; A61K 9/0048; A61P 27/02

(57) ABSTRACT

The present disclosure relates to therapeutic compositions, and more particularly to redox-responsive nanoparticles that allow controlled release of therapeutics, such as vascular endothelial growth factor inhibitors, in body tissues such as in the eye.

### CROSS-REFERENCE TO RELATED APPLICATIONS

This application claims the benefit of priority to U.S. Provisional Application No. 63/042,086, filed Jun. 22, 2020, and U.S. Provisional Application No. 63/055,573, filed Jul. 23, 2020, the disclosure of each of which is incorporated herein by reference in its entirety.

### TECHNICAL FIELD

This disclosure relates to therapeutic compositions, and more particularly to redox-responsive nanoparticles that allow controlled release of therapeutics.

### BACKGROUND

Ophthalmological disorders are often some of the most difficult disorders to treat due to both the delicate nature of the eye as well the inherent issues with proper delivery of therapeutics to specific areas of the eye. Age-related macular degeneration (AMD) is a representative example of an ophthalmological disorder that affected over six million people globally in 2015. AMD is typically divided into a “dry” form and a “wet” form. Currently there are no treatments for dry AMD. Treatment of wet AMD currently relies on monthly intravitreal injection of anti-angiogenic therapeutics to inhibit new choroidal angiogenesis. However, repeated injections have been associated with side effects, are costly, and may lower patient compliance. Moreover, the intraocular oxidative stress mediating angiogenesis is not alleviated by current treatments, which limits the overall efficacy of the treatment strategy. Recently, nanoparticle-based devices present potential in sustained delivery of angiogenesis inhibitors and excellent capability of scavenging reactive oxygen species. Nevertheless, limited efforts have been dedicated to the treatment of AMD via a combined anti-angiogenesis and anti-oxidization pathway. There is a clear need for additional systems for the controlled delivery of drugs for the treatment of ophthalmic disorders.

### SUMMARY

The present disclosure provides drug delivery compositions that allow delivery of therapeutics in a redox-responsive manner, i.e., that allow increased delivery of therapeutic agents upon increased exposure of the composition to increased levels of reactive oxygen species (ROS). In particular, ocular therapeutic compositions are provided that allow redox-responsive release of therapeutics, for example anti-VEGF therapeutics, for the treatment of ophthalmological disorders, for example age-related macular degeneration, cataracts, glaucoma, diabetic retinopathy, or proliferative vitreoretinopathy. The disclosed compositions have been found to both provide sustained released of the particle-bound therapeutics by simple diffusion as well as increased levels of drug release when the particles are exposed to ROS. The drug release from the disclosed compositions is found to undergo two stages: an initial release of therapeutic due to erosion of the particle surface and subsequent diffusion of surface-bound therapeutic; followed by more sustained release of the therapeutic due to simple diffusion of therapeutic bound to the interior of the particles along with increased release due to degradation upon exposure to ROS. The disclosed compositions allow for tailored release of therapeutics directly in response to a pathophysiological indicator of disease state, namely the release of ROS.

Thus in one aspect, an ocular therapeutic composition is provided comprising a population of polydopamine (PDA) nanoparticles; wherein each polydopamine nanoparticle is bound to an anti-vascular endothelial growth factor (VEGF) agent.

In some aspects, the population of PDA nanoparticles has an average particle size ranging from about 10 nm to about 1000 nm, for example from about 100 nm to about 200 nm or from about 120 nm to about 170 nm. In some embodiments, the population of PDA nanoparticles has an average particle size of about 150 nm.

In some embodiments, the anti-VEGF agent comprises an antibody or antibody fragment, for example bevacizumab, ranibizumab or aflibercept. In some embodiments, the anti-VEGF agent comprises an inhibitor of a tyrosine kinase stimulated by VEGF, for example lapatinib, sunitinib, sorafenib, axitinib, or pazopanib.

In some embodiments, each PDA nanoparticle is coated with a polymer. In some embodiments, the polymer comprises an alginate. In some embodiments, the composition further comprising a hydrogel, such as an alginate hydrogel or a hyaluronic acid hydrogel. In some embodiments, the hydrogel comprises poly(N-isopropylacrylamide) grafted sodium hyaluronate hydrogel.

In some embodiments, the anti-VEGF agent is released upon exposure of the PDA nanoparticles to reactive oxygen species (ROS). In some embodiments, the anti-VEGF release upon exposure of the PDA nanoparticles to ROS is due to erosion and/or degradation of the PDA nanoparticles. In some embodiments, the anti-VEGF agent is further released by diffusion out of the PDA nanoparticles.

In another aspect, a method of treating an ophthalmological disorder in a subject in need thereof is provided comprising injecting into the eye of the subject a therapeutically effective amount of an ocular therapeutic composition as described herein. In some embodiments, the ophthalmological disorder is AMD, neovascularization, macular edema, edema, cataract, glaucoma, diabetic retinopathy, proliferative vitreoretinopathy, posterior capsule opacification, presbyopia, and uveitis. In some embodiments, injecting into the eye of the subject comprises injecting into the vitreous chamber of the eye. In other embodiments, injecting into the eye of the subject comprises an intravitreal injection, a subconjunctival injection, a subtenon injection, a retrobulbar injection, or a suprachoroidal injection.

In yet another aspect, a therapeutic composition is provided comprising:
a population of polydopamine (PDA) nanoparticles; wherein each polydopamine nanoparticle is bound to a therapeutic agent;
wherein the therapeutic agent is released upon exposure of the PDA nanoparticle to reactive oxygen species.

In some embodiments, the therapeutic agent release upon exposure of the PDA nanoparticles to ROS is due to erosion and/or degradation of the PDA nanoparticles. In some embodiments, the therapeutic agent may also be further released by simple diffusion out of the PDA nanoparticles, i.e., diffusion not resulting from erosion or degradation of the PDA nanoparticles.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1D depicts the synthesis and characterization of PDA nanoparticles with/without BSA. Drug loaded PDA nanoparticles were successfully prepared according to the increased size based on the TEM images. (FIG. 1A) A schematic illustration for polymerization of dopamine and formation of PDA nanoparticles. (FIG. 1B) UV-Vis spectrum of PDA nanoparticles dispersed in PBS at different concentrations. Increased absorption of PDA nanoparticles was observed as concentration increased. (FIG. 1C) Characteristic TEM images of PDA nanoparticles loaded with/without BSA. (FIG. 1D) The average diameters of PDA nanoparticles with/without BSA determined by TEM images and DLS. After loading BSA, a noticeable increase in nanoparticle size was found.

FIGS. 2A-2B depicts the cell viability of ARPE-19 cells treated by different concentrations of PDA nanoparticles. (FIG. 2A) ARPE-19 cell viability of PDA nanoparticles assessed by MTS assay. Increased concentration of PDA nanoparticles may reduce the cell survival rate. (*=p<0.05) No significant differences in cell viability were observed when treating various concentrations of PDA nanoparticles between 0 to 50 μg/mL (p≥0.05). (FIG. 2B) ARPE-19 cell survival determined by live (green)/dead (red) assay in contact with 0, 1, 100, and 1000 μg/mL PDA nanoparticles dispersed in blank cell media.

FIGS. 3A-3D depicts the degradation of PDA nanoparticles triggered by oxidative species. A clear decrease of particle size after treating the particles with H₂O₂. (FIG. 3A) A schematic illustration of a mechanism for the degradation of PDA in the presence of H₂O₂ and formation of PTCA and PDCA). (FIG. 3B) Characteristic TEM images of PDA nanoparticles treated by 10 mM H₂O₂ for one week. (FIG. 3C) The average diameter of PDA nanoparticles acquired from TEM images before and after the treatment of 10 mM H₂O₂. (FIG. 3B) UV-Vis spectrum of PDA nanoparticles which underwent degradation process induced by different amounts of H₂O₂. A decreased absorption was found in the group of PDA nanoparticles with a higher concentration of H₂O₂, revealing the slow and free radical concentration-dependent degradation of particles (p<0.05).

FIGS. 4A-4E depicts ROS reduction and scavenging by PDA nanoparticles. (FIG. 4A) 10 mM H₂O₂ scavenged by PDA nanoparticles determined by Amplex red assay. A time-dependent decrease in H₂O₂ reacted with PDA nanoparticles was found. A constant decrease in hydrogen peroxide was seen during the first week, confirming scavenging capability of the PDA nanoparticles. (FIG. 4B) Intracellular uptake of 10 μg/mL nanoparticles over 24 hours. Nuclei and F-actin were stained by DAPI (blue/DNA) and phalloidin (red/F-actin), respectively. The PDA nanoparticles were loaded with FITC-BSA (green) for better visualization. (FIG. 4C) Schematic diagram of H₂O₂ and LPS elevating the intracellular oxidative stress which could be indicated by DCFH-DA. (FIG. 4D) Selective fluorescent images of APRE-19 cells with 200 μM H₂O₂ and PDA nanoparticles using DCFH-DA as the fluorescent probe. (FIG. 4E) ROS levels of ARPE-19 cells induced by H₂O₂ or LPS were treated by the various amount of PDA nanoparticles. PDA nanoparticles exhibit good capability in reducing the intracellular ROS. Data (n=3) are presented as mean t standard deviation. A significant difference in fluorescence of the groups treated by PDA nanoparticles (*=p<0.05).

FIGS. 5A-5B depicts the effect of oxidative species on drug release of PDA nanoparticles. FITC-BSA and bevacizumab release profiles triggered by different concentrations of H₂O₂. A higher amount of H₂O₂ led to the faster release of protein therapeutics. Nanoparticles were degraded by 2 months and 3 months in the presence of 10 mM and 5 mM H₂O₂, respectively. The data are presented as average f std. (n=3)

FIGS. 6A-6D depicts the inhibition effects of PDA nanoparticles with/without bevacizumab loaded on HUVECs tube formation under oxidative stress. Cells were presented in green fluorescence (Calcein AM). (FIG. 6A) Scheme of co-culture of HUVECs and ARPE-19 cells under oxidative stress and bevacizumab loaded PDA nanoparticles inhibiting capillary structure development. (FIG. 6B) The amount of VEGF production in the groups of PDA nanoparticles and bevacizumab under 200 μM H₂O₂ determined by VEGF ELISA. A significant decrease of VEGF was recognized in groups treated by nanoparticles and bevacizumab. (*=p<0.05) (FIG. 6C) Mean HUVECs tube length of groups treated with none, 200 μM H₂O₂, 10 μg/mL PDA nanoparticles+200 μM H₂O₂, 10 μg/mL bevacizumab loaded PDA nanoparticles+200 μM H₂O₂, and 5 μg/mL free native bevacizumab (Bev)+200 μM H₂O₂ analyzed by ImageJ. A significant difference in HUVEC tube length of the group treated by PDA nanoparticles and bevacizumab. (*=p<0.05) (FIG. 6D) Representative fluorescent images showing HUVECs of the indicated groups. (Scale bar=300 μm) A significant tube disruption is found in groups with positive control.

FIGS. 7A-7B depicts the intravitreal injection feasibility of PDA nanoparticles via a 31-gauge needle. (FIG. 7A) schematic diagram presenting the intravitreal injection of particles into ex vivo model. (FIG. 7B) 100 μL PDA nanoparticles were injected into freshly enucleated porcine eyes. The particles were in black color and successfully delivered to the vitreous humor.

Like reference symbols in the various drawings indicate like elements.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an ocular therapeutic composition,” “a therapeutic agent,” or “a clinical condition,” includes, but is not limited to, two or more such ocular therapeutic compositions, therapeutic agents, or clinical conditions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y”’, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

It is understood that disclosure herein of a therapeutic agent also disclosed pharmaceutically acceptable salt, pharmaceutically acceptable ester, pharmaceutically acceptable amide, prodrug forms, and derivates of the therapeutic agent.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to: sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software.

### Polydopamine Nanoparticles

In one aspect, the compositions of the present invention comprise a population of polydopamine nanoparticles.

Polydopamine is formed by the oxidation of dopamine. It is biomimetic of the proteins on the extremity of mussel byssus which are extremely right in L-DOPA and L-Lysine residues. These amino acid residues, containing catechol and amino functional groups, allow for strong adhesion of the mussel to all kinds of substrates in the wet and slightly basic environment of sea water. Because of this, polydopamine has traditionally found extensive use in adhesive coatings. Methods for forming polydopamine nanoparticles are known in the art. Typically, polydopamine nanoparticles are formed alkaline aqueous solutions (for example, in the presence of Tris buffer at pH=8.5) in the presence of an oxidant. Oxygen dissolved in the aqueous solution is typically used as the oxidant, but other oxidants may be used, for example ammonium peroxodisulfate or sodium periodate. In some embodiments, the polydopamine nanoparticles as used in the present disclosure are essentially spherical, spheroid, ellipsoid, or combinations thereof.

In some embodiments, the polydopamine particles as used in the present disclosure have an average particle size ranging from about 10 nm to about 1000 nm, for example from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 900 nm to about 1000 nm, from about 10 nm to about 900 nm, from about 100 nm to about 900 nm, from about 200 nm to about 900 nm, from about 300 nm to about 900 nm, from about 400 nm to about 900 nm, from about 500 nm to about 900 nm, from about 600 nm to about 900 nm, from about 700 nm to about 900 nm, from about 800 nm to about 900 nm, from about 10 nm to about 800 nm, from about 100 nm to about 800 nm, from about 200 nm to about 800 nm, from about 300 nm to about 800 nm, from about 400 nm to about 800 nm, from about 500 nm to about 800 nm, from about 600 nm to about 800 nm, from about 700 nm to about 800 nm, from about 10 nm to about 700 nm, from about 100 nm to about 700 nm, from about 200 nm to about 700 nm, from about 300 nm to about 700 nm, from about 400 nm to about 700 nm, from about 500 nm to about 700 nm, from about 600 nm to about 700 nm, from about 10 nm to about 600 nm, from about 100 nm to about 600 nm, from about 200 nm to about 600 nm, from about 300 nm to about 600 nm, from about 400 nm to about 600 nm, from about 500 nm to about 600 nm, from about 10 nm to about 500 nm, from about 100 nm to about 500 nm, from about 200 nm to about 500 nm, from about 300 nm to about 500 nm, from about 400 nm to about 500 nm, from about 10 nm to about 400 nm, from about 100 nm to about 400 nm, from about 200 nm to about 400 nm, from about 300 nm to about 400 nm, from about 10 nm to about 300 nm, from about 100 nm to about 300 nm, from about 200 nm to about 300 nm, from about 10 nm to about 200 nm, from about 100 nm to about 200 nm, or from about 10 nm to about 100 nm.

In some embodiments, the population of polydopamine nanoparticles has an average particle size of about 10 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm.

In some embodiments, the polydopamine particles as described herein may be further comprise a coating. The coating can be disposed on the surface of the particle, for example by bonding, adsorption or by complexation. The coating can also be intermingled or dispersed within the particle as well as disposed on the surface of the particle.

In some embodiments, the coating may comprise a polymer, i.e., the polydopamine particles as described herein may be coated with a polymer. In some embodiments, the polymer may comprise an alginate. In some embodiments, the polymer may comprise polyethylene glycol, polyvinyl alcohol, or similar substances. In some embodiments, the coating may also comprise a non-ionic surfactant such as those composed of polyalkylene oxide, e.g. polyethylene glycol or polypropylene glycol, and can include a copolymer of more than one alkylene oxide. In some embodiments, the coating can comprise a polyoxyethylene-polyoxypropylene copolymer, i.e., a poloxamer such a poloxamer 188, 237, 338, and 407.

In some embodiments, the polydopamine nanoparticles have a loading efficiency of the therapeutic of greater than 0.1%, for example greater than 0.5%, greater than 1%, greater than 5%, greater than 10%, greater than 15%, greater than 20%, greater than 25%, greater than 30%, greater than 35%, greater than 40%, greater than 45%, or greater than 50%. In some embodiments, the therapeutic agent (for example, the anti-VEGF therapeutic agent) may be loaded in the polydopamine nanoparticles in an amount of about 10 μg per mg, about 20 μg per mg, about 30 μg per mg, about 40 μg per mg, about 50 μg per mg, about 60 μg per mg, about 70 μg per mg, about 80 μg per mg, about 90 μg per mg, about 100 μg per mg, about 110 μg per mg, about 120 μg per mg, about 130 μg per mg, about 140 μg per mg, about 150 μg per mg, about 160 μg per mg, about 170 μg per mg, about 180 μg per mg, about 190 μg per mg, about 200 μg per mg, about 225 μg per mg, about 250 μg per mg, about 275 μg per mg, about 300 μg per mg, about 325 μg per mg, about 350 μg per mg, about 375 μg per mg, about 400 μg per mg, about 425 μg per mg, about 450 μg per mg, about 475 μg per mg, about 500 μg per mg, or more.

### Anti-VEGF Therapeutic Agents

In some aspects of the ocular therapeutic composition, each PDA nanoparticle within the population of PDA nanoparticles is bound to an anti-vascular endothelial growth factor (VEGF) agent.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-ANPF gene including VEGF121, VEGF165 and VEGF189. Further, as used herein, the term “VEGF” includes VEGF-related angiogenic factors such as PIGF (placental growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1), VEGFR-2 (KDR/Flk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF165. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

Representative examples of anti-VEGF agents include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLTO1, sFLT02, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C (VEGF-C), human vascular endothelial growth factor-D (VEGF-D) and human vascular endothelial growth, factor-E (VEGF-E), and an antibody that binds, to an epitope of VEGF.

In various aspects, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three dimensional structure of VEGF that is displayed, such that the epitope is exposed on the surface of the folded VEGF molecule. In one embodiment, the epitope is a linear amino acid sequence from VEGF.

In various aspects, the anti-VEGF agent may comprise an agent that blocks or inhibits VEGF-mediated activity, e.g., one or more VEGF antisense nucleic acids. The present disclosure provides the therapeutic or prophylactic use of nucleic acids comprising at least six nucleotides that are antisense to a gene or cDNA encoding VEGF or a portion thereof. As used herein, a VEGF “antisense” nucleic acid refers to a nucleic acid capable of hybridizing by virtue of some sequence complementarity to a portion of an RNA (preferably mRNA) encoding VEGF. The antisense nucleic acid may be complementary to a coding and/or noncoding region of an mRNA encoding VEGF. Such antisense nucleic acids have utility as compounds that prevent VEGF expression, and can be used in the treatment of diabetes. The antisense nucleic acids of the disclosure are double-stranded or single-stranded oligonucleotides, RNA or DNA or a modification or derivative thereof, and can be directly administered to a cell or produced intracellularly by transcription of exogenous, introduced sequences.

The VEGF antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double-stranded RNAs, known as siRNAs, and short hairpin RNAs, and long dsRNA (>50 bp but usually ≥500 bp).

In various aspects, the anti-VEGF agent may comprise one or more ribozyme molecule designed to catalytically cleave gene mRNA transcripts encoding VEGF, preventing translation of target gene mRNA and, therefore, expression of the gene product.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA and must include the well-known catalytic sequence responsible for mRNA cleavage. For this sequence, see, e.g., U.S. Pat. No. 5,093,246. While ribozymes that cleave mRNA at site-specific recognition sequences can be used to destroy mRNAs encoding VEGF, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present disclosure also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place. The disclosure encompasses those Cech-type ribozymes that target eight base-pair active site sequences that are present in the gene encoding VEGF.

In further aspects, the anti-VEGF agent may comprise an antibody that inhibits VEGF such as bevacizumab, ranibizumab or aflibercept. In still further aspects, the anti-VEGF agent may comprise an agent that inhibits VEGF activity such as a tyrosine kinases stimulated by VEGF, examples of which include, but are not limited to lapatinib, sunitinib, sorafenib, axitinib, and pazopanib.

In some embodiments, the anti-VEGF agent is bound within the PDA nanoparticle, i.e., as a result encapsulation within PDA nanoparticle upon assembly. In some embodiments, the anti-VEGF agent is bound to the surface of the PDA nanoparticle. In some embodiments, the anti-VEGF agent is both bound to the surface of and bound within the PDA nanoparticle.

In some embodiments, the anti-VEGF agent is released upon exposure of the PDA nanoparticles to reactive oxygen species (ROS). In some embodiments, the release upon exposure of the PDA nanoparticles to ROS is due to erosion and/or degradation of the PDA nanoparticles. “Erosion” refers to the chemical decomposition of the PDA polymer on the surface of the PDA nanoparticles. Erosion may lead to diffusion of any anti-VEGF agents adsorbed to the surface of the PDA nanoparticle. “Degradation” refers to chemical decomposition of PDA polymer found on the interior of the PDA nanoparticles. In some embodiments, the anti-VEGF agent is further released by diffusion out of the PDA nanoparticles.

In some embodiments, the PDA nanoparticles are characterized by an initial rapid release of a portion of bound anti-VEGF agent followed by a longer sustained release. While not wishing to be bound by any theory, the initial rapid release (which typically occurs within approximately the first two weeks of implantation of the particles) may result from the initial diffusion of surface-bound anti-VEGF agent upon initial erosion of the PDA nanoparticle. The longer sustained release (lasting for about 1 month, 2 months, 3 months, 4 months, 5 months, 6 months or more) may be the result of simple diffusion of the anti-VEGF agent from the interior of the PDA nanoparticles and/or may also result from degradation of the PDA nanoparticle interior by exposure to ROS.

### Ocular Therapeutic Compositions

In one aspect of the present disclosure, an ocular therapeutic composition is provided comprising a population of polydopamine (PDA) nanoparticles; wherein each polydopamine nanoparticle is bound to an anti-vascular endothelial growth factor (VEGF) agent. The precise formulation of the ocular therapeutic composition will vary according to a wide range of commercial and scientific criteria. Thus, one of skill in the art will appreciate that the above disclosed formulation may contain additional agents.

For example, the ocular therapeutic compositions described herein may be prepared using a physiological saline solution as a vehicle. The pH of the ocular therapeutic composition may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

Any diluent used in the preparation of the ocular therapeutic compositions may preferably be selected so as not to unduly affect the biological activity of the composition. Example of such diluents which are especially for injectable ophthalmic compositions are water, various saline, organic, or inorganic salt solutions, Ringer's solution, dextrose solution, and Hank's solution.

In addition, the ocular therapeutic compositions may include additives such other buffers, diluents, carriers, adjuvants, or excipients. Any pharmaceutically acceptable buffer suitable for application to the eye may be used, e.g., tris or phosphate buffers. Other agent may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80.

Other vehicles that may be used include, but are not limited to, polyvinyl alcohol, povidone, hydroxypropyl methyl cellulose, poloxamers, carboxymethyl cellulose, hydroxyethyl cellulose, or purified water. Tonicity adjustors may be included, for example, sodium chloride, potassium chloride, mannitol, or glycerin. Antioxidants include, but are not limited to, sodium metabisulfite, sodium thiosulfate, acetylcysteine, butylated hydroxyanisole, or butylated hydroxytoluene. The indications, effective doses, formulations, contraindications, etc. of the components in the ophthalmic composition are available and are known to one skilled in the art.

These agents may be present in individual amounts from about 0.001% to about 5% by weight and preferably about 0.01% to about 2% by weight in the formulation. Suitable water soluble buffering agents that may be employed are sodium carbonate, sodium borate, sodium phosphate, sodium acetate, or sodium bicarbonate, as approved by the U.S. FDA for the desired route of administration. These agents may be present in amounts sufficient to maintain a pH of the system between about 2 to about 9 and preferably about 4 to about 8. As such, the buffering agent may be as much as about 5% (w/w) of the total ocular therapeutic composition. Electrolytes such as, but limited to, sodium chloride and potassium chloride may be also included in the formulation.

In some embodiments, the ocular therapeutic composition further comprises a hydrogel. In some embodiments, the hydrogel comprises a polymer composition, for example a homopolymer, a copolymer, or combinations thereof. In some embodiments, the hydrogel comprises one or more hydrophilic polymers, i.e. a polymer having at least 0.1 wt. % solubility in water, for example having at least 0.5 wt. % solubility. In some embodiments, the hydrophilic polymer has a solubility of at least 1 mg/mL.

In some embodiments, the polymer composition may comprise one or more vinyl alcohol residues. In some embodiments, the polymer composition may comprise one or more acrylamide residues. In some embodiments, the polymer composition may comprise one or more residues selected from a polyethylene glycol derivative or a functionalized polyethylene glycol. In some embodiments, the polymer composition may comprise one or more acrylate residues or one or more methacrylate residues. In some embodiments, the polymer composition may comprise one or more residues selected from acrylamide, N-ornithine acrylamide, N-(2-hydroxypropyl)acrylamide, hydroxyethylacrylate, hydroxyethylmethacrylate, polyethyleneglycol acrylates, polyethylenegylcol methacrylates, N-vinylpyrrolidinone, N-phenylacrylamide, dimethylaminopropyl methacrylamide, acrylic acid, benzylmethacrylamide, methylthioethylacrulamide, or combinations thereof.

Representative examples of hydrogels which can be used include, but are not limited to, hyaluronic acid, collagen, gellan, silk, fibrin, alginate, chitosan, polyacrylamides and methacrylate derivatives thereof, polyacrylic acid and methacrylate derivatives thereof, polyvinyl alcohol, polyethylene glycol and derivatives thereof, polypropylene glycol and derivatives thereof, or combinations thereof.

In some embodiments, the hydrogel comprises a hyaluronate derivative, for example poly(N-isopropylacrylamide) grafted sodium hyaluronate.

### Other Therapeutic Agents

In another aspect of the present disclosure, a therapeutic composition is provided comprising.
a population of polydopamine (PDA) nanoparticles; wherein each polydopamine nanoparticle is bound to a therapeutic agent; and
wherein the therapeutic agent is released upon exposure of the PDA nanoparticle to reactive oxygen species (ROS).

In some embodiments, the therapeutic agent is bound within the PDA nanoparticle, i.e., as a result encapsulation within PDA nanoparticle upon assembly. In some embodiments, the therapeutic agent is bound to the surface of the PDA nanoparticle. In some embodiments, the therapeutic agent is both bound to the surface of and bound within the PDA nanoparticle.

In some embodiments, the release upon exposure of the PDA nanoparticles to ROS is due to erosion and/or degradation of the PDA nanoparticles. In some embodiments, the therapeutic agent is further released by simple diffusion out of the PDA nanoparticles, i.e., diffusion not resulting from erosion or degradation of the PDA nanoparticles.

In some embodiments, the PDA nanoparticles are characterized by an initial rapid release of a portion of bound therapeutic agent followed by a longer sustained release. While not wishing to be bound by any theory, the initial rapid release (which typically occurs within approximately the first two weeks of implantation of the particles) may result from the initial diffusion of surface-bound therapeutic agent upon initial erosion of the PDA nanoparticle. The longer sustained release (lasting for about 1 month, 2 months, 3 months, 4 months, 5 months, 6 months or more) may be the result of simple diffusion of the therapeutic agent from the interior of the PDA nanoparticles and/or may also result from degradation of the PDA nanoparticle interior by exposure to ROS.

As used herein, a “therapeutic agent” refers to one or more therapeutic agents, active ingredients, or substances that can be used to treat a medical condition of the eye or a cancer. The therapeutic agents are typically ophthalmically acceptable and are provided in a form that does not cause adverse reactions when the compositions disclosed herein are placed in an eye. As discussed herein, the therapeutic agents can be released from the disclosed compositions in a biologically active form. For example, the therapeutic agents may retain their three-dimensional structure when released from the composition into an eye.

It is further understood, that as used herein, the terms “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

In some embodiments, the therapeutic agent may comprise an agent useful in the treatment of an ophthalmological disorder or an eye disease such as: beta-blockers including timolol, betaxolol, levobetaxolol, and carteolol; miotics including pilocarpine; carbonic anhydrase inhibitors; serotonergics; muscarinics; dopaminergic agonists; adrenergic agonists including apraclonidine and brimonidine; anti-angiogenesis agents; anti-infective agents including quinolones such as ciprofloxacin and aminoglycosides such as tobramycin and gentamicin; non-steroidal and steroidal anti-inflammatory agents, such as suprofen, diclofenac, ketorolac, rimexolone and tetrahydrocortisol; growth factors, such as EGF; immunosuppressant agents; and anti-allergic agents including olopatadine; prostaglandins such as latanoprost; 15-keto latanoprost; travoprost; and unoprostone isopropyl.

In some embodiments, the therapeutic agent comprises an anti-VEGF agent as described herein.

In some embodiments, the therapeutic agent is selected from the group consisting of an anti-inflammatory agent, a calcineurin inhibitor, an antibiotic, a nicotinic acetylcholine receptor agonist, and an anti-lymphangiogenic agent. In some embodiments, the anti-inflammatory agent may be cyclosporine. In some embodiments, the calcineurin inhibitor may be voclosporin. In some embodiments, the antibiotic may be selected from the group consisting of amikacin, gentamycin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline. In some embodiments, the nicotinic acetylcholine receptor agonist may be any of pilocarpine, atropine, nicotine, epibatidine, lobeline, or imidacloprid. In some embodiments, the anti-lymphangiogenic agent may be a vascular endothelial growth factor C (VEGF-C) antibody, a VEGF-D antibody or a VEGF-3 antibody.

In some aspects, the therapeutic agent may be selected from: a beta-blocker, including levobunolol (BETAGAN), timolol (BETIMOL, TIMOPTIC), betaxolol (BETOPTIC) and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine; a steroid derivative, such as 2-methoxyestradiol or analogs or derivatives thereof; or an antibiotic.

The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

In some embodiments, the therapeutic agent may comprise a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non limiting examples of renin inhibitors which may be used as therapeutic agents include, but are not limited to: aliskiren, ditekiren, enalkiren, remikiren, terlakiren, ciprokiren and zankiren, pharmaceutically acceptable salts thereof, and mixtures thereof.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others. In some embodiments, the therapeutic agent may comprise a steroid.

The terms “peroxisome proliferator-activated receptor gamma agent,” or “PPAR-γ agent,” or “PPARG agent,” or “PPAR-gamma agent” refers to agents which directly or indirectly act upon the peroxisome proliferator-activated receptor. This agent may also influence PPAR-alpha, “PPARA” activity.

In some embodiments, the therapeutic agent may comprise a modulator of macrophage polarization. Illustrative modulators of macrophage polarization include peroxisome proliferator activated receptor gamma (PPAR-g) modulators, including, for example, agonists, partial agonists, antagonists or combined PPAR-gamma/alpha agonists. In some embodiments, the therapeutic agent may comprise a PPAR gamma modulator, including PPAR gamma modulators that are full agonists or a partial agonists. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, and rhodanine. In some embodiments, the PPAR gamma modulator is one or more of irbesartan and telimesartan. In some embodiments, the PPAR gamma modulator is a nonsteroidal anti-inflammatory drug (NSAID, such as, for example, ibuprofen) or an indole. Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11):750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In some embodiments, the therapeutic agent may comprise semapimod (CNI-1493) as described in Bianchi, et al. (March 1995). Molecular Medicine (Cambridge, Mass.) 1 (3): 254-266, the contents of which is hereby incorporated by reference in its entirety.

In some embodiments, the therapeutic agent may comprise a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S,R)-3-(4-hydroxyphenyl)-4,5-dihydro-5-isoxazole acetic acid methyl ester (ISO-1), isoxazoline, p 425 (J. Biol. Chem., 287, 30653-30663), epoxyazadiradione, or vitamin E.

In some embodiments, the therapeutic agent may comprise a chemokine receptor 2 (CCR2) inhibitor as described in, for example, U.S. patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (PLOS ONE 7(3): e32864).

In some embodiments, the therapeutic agent may comprise an agent that modulates autophagy, microautophagy, mitophagy or other forms of autophagy. In some embodiments, the therapeutic agent may comprise sirolimus, tacrolimis, rapamycin, everolimus, bafilomycin, chloroquine, hydroxychloroquine, spautin-1, metformin, perifosine, resveratrol, trichostatin, valproic acid, Z-VAD-FMK, or others known to those in the art. Without wishing to be bound by theory, agent that modulates autophagy, microautophagy, mitophagy or other forms of autophagy may alter the recycling of intra-cellular components, for example, but not limited to, cellular organelles, mitochondria, endoplasmic reticulum, lipid or others. Without further wishing to be bound by theory, this agent may or may not act through microtubule-associated protein 1A/1B-light chain 3 (LC3).

According to certain aspects, the therapeutic agent may comprise a biologic drug, particularly an antibody. According to some aspects, the antibody is selected from the group consisting of cetuximab, anti-CD24 antibody, panitumumab and bevacizumab.

Therapeutic agents as used in the present disclosure may comprise peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may comprise CNS-active drugs, neuro-active drugs, inflammatory and anti-inflammatory drugs, renal and cardiovascular drugs, gastrointestinal drugs, anti-neoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians' Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIH, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomegalovirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12,13, 15, 16, 17 and 18.

In some embodiments, the therapeutic agent comprises a drug used in the treatment of cataract or posterior capsule opacification. In some embodiments, the therapeutic agent comprises a drug used in the treatment of proliferative vitreoretinopathy. In some embodiments, the therapeutic agent comprises a drug used in the treatment of diabetic retinopathy, for example insulin. In some embodiments, the therapeutic agents comprises a drug used in the treatment of uveal melanoma, for example dacarbazine, interferon-alpha, cisplatin, tamoxifen, sunitinib, fotemustine, crizotinib, valproic acid, ipilimumab, nivolumab, or combinations thereof.

### Method of Treatment Using Disclosed Therapeutic Compositions

Methods of treating a clinical condition by administration of a disclosed therapeutic composition are also provided herein. A clinical condition can be a clinical disorder, disease, dysfunction or other condition that can be ameliorated by a therapeutic composition.

The term “administering” or “administration” of a disclosed therapeutic composition to a subject includes any route of introducing or delivering to a subject the device to perform its intended function. Administration can be carried out by any suitable route, including orally, intranasally, parenterally (intravenously, intramuscularly, intraperitoneally, or subcutaneously), or topically. Administration includes self-administration and the administration by another. In some instances, administration is via injection to the eye, including intraocular injection.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. The term “sequential” therefore is different than “simultaneous” administration.

The term “simultaneous” administration refers to the administration of at least two active ingredients by the same route at the same time or at substantially the same time.

The term “therapeutic” as used herein means a treatment and/or prophylaxis. A therapeutic effect is obtained by suppression, remission, or eradication of a disease state.

The present disclosure further provides methods of treating an ophthalmological disease or disorder by administering a therapeutically effective amount of the ocular therapeutic compositions described herein. In some embodiments, the disclosed methods pertain to treatment of an ophthalmological disorder comprising injecting a therapeutically effective amount of the disclosed ocular therapeutic composition into the eye of a subject. The subject can be a patient; and the patient can have been diagnosed with an ophthalmological disorder. In some aspects, the method can further comprise diagnosing a subject with an ophthalmological disorder.

The ophthalmological disorder can be acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion, a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, a cancer, and glaucoma. In certain instances, the ophthalmological disorder is wet age-related macular degeneration (wet AMD), a cancer, neovascularization, macular edema, or edema. In a further particular aspect, the ophthalmological disorder is wet age-related macular degeneration (wet AMD).

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

“Ocular region” or “ocular site” means any area of the ocular globe (eyeball), including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include, but are not limited to, the anterior chamber, the posterior chamber, the vitreous cavity, the choroid, the suprachoroidal space, the conjunctiva, the subconjunctival space, the episcleral space, the intracorneal space, the subretinal space, sub-Tenon's space, the epicorneal space, the sclera, the pars plana, surgically-induced avascular regions, the macula, and the retina.

“Ophthalmological disorder” can mean a disease, ailment or condition which affects or involves the eye or one of the parts or regions of the eye. Broadly speaking, the eye includes the eyeball, including the cornea, and other tissues and fluids which constitute the eyeball, the periocular muscles (such as the oblique and rectus muscles) and the portion of the optic nerve which is within or adjacent to the eyeball.

“Glaucoma” means primary, secondary and/or congenital glaucoma. Primary glaucoma can include open angle and closed angle glaucoma. Secondary glaucoma can occur as a complication of a variety of other conditions, such as injury, inflammation, pigment dispersion, vascular disease and diabetes. The increased pressure of glaucoma causes blindness because it damages the optic nerve where it enters the eye. Thus, in one nonlimiting embodiment, by lowering reactive oxygen species, STC-1, or MSCs which express increased amounts of STC-1, may be employed in the treatment of glaucoma and prevent or delay the onset of blindness.

Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic retinopathy, uveitis, proliferative vitreoretinopathy (PVR), sympathetic ophthalmia, Vogt-Koyanagi-Harada (VKH) syndrome, histoplasmosis, and uveal diffusion.

“Injury” or “damage” in relation to an ocular condition are interchangeable and refer to the cellular and morphological manifestations and symptoms resulting from an inflammatory-mediated condition, such as, for example, inflammation, as well as tissue injuries caused by means other than inflammation, such as chemical injury, including chemical burns, as well as injuries caused by infections, including but not limited to, bacterial, viral, or fungal infections.

“Intraocular” means within or under an ocular tissue. An intraocular administration of an ocular therapeutic composition includes administration of the ocular therapeutic composition to a sub-tenon, subconjunctival, suprachoroidal, subretinal, intravitreal, anterior chamber, and the like location. An intraocular administration of an ocular therapeutic composition excludes administration of the drug delivery system to a topical, systemic, intramuscular, subcutaneous, intraperitoneal, and the like location.

“Macular degeneration” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and capillary endothelial cells. Age-related macular degeneration, or ARMD, is the major macular degeneration related condition, but a number of others are known including, but not limited to, Best macular dystrophy, Stargardt macular dystrophy, Sorsby fundus dystrophy, Mallatia Leventinese, Doyne honeycomb retinal dystrophy, and RPE pattern dystrophies. Age-related macular degeneration (AMD) is described as either “dry” or “wet.” The wet, exudative, neovascular form of AMD affects about 10-20% of those with AMD and is characterized by abnormal blood vessels growing under or through the retinal pigment epithelium (RPE), resulting in hemorrhage, exudation, scarring, or serous retinal detachment. Eighty to ninety percent of AMD patients have the dry form characterized by atrophy of the retinal pigment epithelium and loss of macular photoreceptors. Drusen may or may not be present in the macula. There may also be geographic atrophy of retinal pigment epithelium in the macula accounting for vision loss. At present there is no cure for any form of AMD, although some success in attenuation of wet AMD has been obtained with photodynamic and especially anti-VEGF therapy.

“Drusen” is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts. There is no generally accepted therapeutic method that addresses drusen formation and thereby manages the progressive nature of AMD.

“Ocular neovascularization” (ONV) is used herein to refer to choroidal neovascularization or retinal neovascularization, or both.

“Retinal neovascularization” (RNV) refers to the abnormal development, proliferation, and/or growth of retinal blood vessels, e.g., on the retinal surface.

“Subretinal neovascularization” (SRNVM) refers to the abnormal development, proliferation, and/or growth of blood vessels beneath the surface of the retina.

“Cornea” refers to the transparent structure forming the anterior part of the fibrous tunic of the eye. It consists of five layers, specifically: 1) anterior corneal epithelium, continuous with the conjunctiva; 2) anterior limiting layer (Bowman's layer); 3) substantia propria, or stromal layer; 4) posterior limiting layer (Descemet's membrane); and 5) endothelium of the anterior chamber or keratoderma.

“Retina” refers to the innermost layer of the ocular globe surrounding the vitreous body and continuous posteriorly with the optic nerve. The retina is composed of layers including the: 1) internal limiting membrane; 2) nerve fiber layer; 3) layer of ganglion cells; 4) inner plexiform layer; 5) inner nuclear layer; 6) outer plexiform layer; 7) outer nuclear layer; 8) external limiting membrane; and 9) a layer of rods and cones.

“Retinal degeneration” refers to any hereditary or acquired degeneration of the retina and/or retinal pigment epithelium. Non-limiting examples include retinitis pigmentosa, Best's Disease, RPE pattern dystrophies, and age-related macular degeneration.

In various aspects, a method of treating an ophthalmological disorder may comprise treatment of various ocular diseases or conditions of the retina, including the following: maculopathies/retinal degeneration; macular degeneration, including age-related macular degeneration (ARMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, and intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e., front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; posterior capsule opacification (PCO); conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Other diseases or disorders of the eye which may be treated in accordance with the present invention include, but are not limited to, ocular cicatricial pemphigoid (OCP), Stevens Johnson syndrome and cataracts.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e., the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic retinopathy; uveitis; ocular histoplasmosis; infections, such as fungal or viral-caused infections; macular degeneration, such as acute macular degeneration, non-exudative age-related macular degeneration and exudative age-related macular degeneration; edema, such as macular edema, cystoid macular edema and diabetic macular edema; multifocal choroiditis; ocular trauma which affects a posterior ocular site or location, ocular tumors; retinal disorders, such as central retinal vein occlusion, diabetic retinopathy (including proliferative diabetic retinopathy), proliferative vitreoretinopathy (PVR), retinal arterial or venous occlusive disease, retinal detachment, uveitic retinal disease; sympathetic ophthalmia; Vogt-Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal ganglion cells or retinal nerve fibers (i.e., neuroprotection).

In some embodiments, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation, vitrectomy, corneal transplantation, forms of lamellar keratectomy (DSEK, etc.), and radial keratotomy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

### Kits

The present disclosure also pertains to kits comprising one of: (a) the ocular therapeutic composition as described herein; (b) the ocular therapeutic composition as described herein in a sterile package; or (c) a pre-filled syringe or needle comprising the ocular therapeutic composition as described herein; and instructions for administering the ocular therapeutic composition as described herein to treat a clinical condition or pathology.

In a further aspect, the disclosed kits can be packaged in a daily dosing regimen (e.g., packaged on cards, packaged with dosing cards, packaged on blisters or blow-molded plastics, etc.). Such packaging promotes products and increases ease of use for administration by a health care profession. Such packaging can also reduce potential medical errors. The present invention also features such kits further containing instructions for use.

In a further aspect, the present disclosure also provides a pharmaceutical pack or kit comprising one or more packages comprising the disclosed ocular therapeutic composition. Associated with such packages can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise further therapeutic agents, compounds and/or products co-packaged, co-formulated, and/or co-delivered with other components. For example, a drug manufacturer, a drug reseller, a physician, a compounding shop, or a pharmacist can provide a kit comprising a disclosed ocular therapeutic composition and another component for delivery to a patient.

It is contemplated that the disclosed kits can be used in connection with the disclosed methods of making, the disclosed methods of using or treating, and/or the disclosed compositions.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Controlled Release of Anti-VEGF by Redox-Responsive Polydopamine Nanoparticles

Reactive oxidative species (ROS) are the primary mediator of angiogenesis by upregulating the expression of vascular endothelial growth factor in the development of wet age-related macular degeneration (AMD). However, the current treatment of AMD currently relies on monthly intravitreal injection of anti-angiogenic therapeutics to inhibit new choroidal angiogenesis. However, repeated injections have been associated with side-effects, are costly, and may lower patient compliance. Moreover, the intraocular oxidative stress-dependent angiogenesis is not alleviated by current treatments, which limits the overall efficacy of the treatment strategy. Recently, nanoparticle-based devices present potential in sustained delivery of angiogenesis inhibitors and excellent capability of scavenging reactive oxygen species. Nevertheless, limited efforts have been dedicated to the treatment of oxidative stress-related diseases via a combined anti-angiogenesis and anti-oxidization pathway. For this purpose, anti-angiogenetic protein-loaded polydopamine (PDA) nanoparticles were developed for the enhanced treatment of AMD. Remarkably, the PDA nanoparticles could efficiently scavenge reactive oxidative species (ROS) to reduce the expression of angiogenic agents. In parallel, the particles were able to controllably release loaded anti-angiogenic drugs in response to oxidative stress.

### BACKGROUND

Angiogenesis, the formation of new blood vessels, is a fundamental physiological process for growth and development.^{1, 2} However, abnormal angiogenesis has been associated with a variety of diseases such as cancer, retinopathy, as well as wet age-related macular degeneration (AMD).^{3, 4} Although several therapeutics targeting abnormal angiogenesis have been clinically used, most of them have only been able to slow down the angiogenesis process, leaving several limitations. For example, wet AMD is the fourth most common ocular disease which may lead to permanent blindness if left untreated. There is currently no way to prevent or cure AMD, so clinicians try to prevent disease progression^{5, 6} The current gold standard of treating wet AMD is the monthly intravitreal administration of anti-VEGF compounds, such as bevacizumab, ranibizumab, or aflibercept, to block free active vascular endothelial growth factor (VEGF) from initiating choroidal vascularization (CNV).⁷ However, this treatment often requires monthly intravitreal injection, which can be associated with low patient compliance and increased risk of clinical complications.⁸ Therefore, it is crucial to develop an effective delivery platform to sustainably release anti-VEGF and reduce injection frequency, thereby improving the reliability and patient compliance of the anti-VEGF-based suppression of angiogenesis.

Another critical barrier for suppressing angiogenesis is the relatively high level of reactive oxidative species (ROS) often existent in the microenvironment at the sites of disease (e.g. tumor growth and AMD).⁴ ROS generated during cell respiration and metabolism are primary mediators regulating various cellular functions.⁹ However, these ROS are able to induce angiogenesis by upregulating the expression of hypoxia-inducible factor-1 (HIF-1) and VEGF.¹⁰⁻¹² The development of various diseases has been known to be closely related to ROS-dependent angiogenesis. A broad consensus on the pathogenesis of wet AMD is that ROS increase the expression of VEGF and further result in CNV.^{13, 14} This angiogenesis eventually contributes to severe and irreversible retinal damage as the abnormal blood vessels bleed and scar.^{12, 15, 16} As such, there is an urgent need for developing reliable strategies to: i) reduce ROS levels; and ii) improve the delivery of anti-VEGF, to achieve efficient anti-angiogenesis for improving the treatment of AMD and other diseases.

To this end, a polydopamine (PDA) nanoparticle-based redox-responsive protein delivery system for enhanced anti-angiogenesis therapy is described in this example. More specifically, the described bi-functional delivery system can: i) load anti-VEGF therapeutics; ii) release anti-VEGF in a sustainable and ROS-responsive manner; iii) robustly reduce the oxidative stress of retinal cells through a unique redox mechanism; and iv) biodegrade in a controlled manner. Although several drug delivery systems have been recently investigated to extend the release period of anti-VEGF, ocular drug delivery devices built upon these biomaterials are still limited by short dosing intervals.^{8, 17, 18} The hydrophobic and π-π interactions between anti-VEGF proteins and aromatic structures in the PDA nanoparticles was utilized to improve release period and loading rate of anti-VEGF. This is important for ocular diseases which require long-term drug administration such as AMD, where frequent intraocular injections of anti-VEGF can potentially lead to various adverse effects.¹⁹⁻²¹

Additionally, the synergistic anti-angiogenesis by a combined reduction of ROS and VEGF is essential in efficiently treating oxidative stress-dependent diseases. Recent reports have shown that PDA was able to inhibit acute inflammation by scavenging ROS and capturing metal ions, possibly due to the abundant phenol groups.^{15, 20, 22-25} More specifically, PDA degrades via redox reaction in the presence of oxidative species which then alleviates oxidative stress.¹⁹ While there have been other anti-oxidant compounds investigated for scavenging intraocular ROS clinically, such as multivitamins, most of them have been administrated orally at high doses and have low bioavailability, limiting their efficacy for treating CNV at a later stage of AMD.^{26, 27} Several metal-oxides, such as ceria oxides, have also been studied for their capability of scavenging free radicals and slowing down the progression of AMD.^{28, 29} However, concerns on their biocompatibility have significantly restricted their clinical applications.³⁰ Building upon previous work, biocompatible and biodegradable PDA was selected to be investigated as a unique solution for reducing ROS in the microenvironment of AMD as well as other diseases.

With these considerations, in this example anti-VEGF loaded PDA nanoparticles were synthesized through in-situ encapsulation. As a proof of concept, bevacizumab, a full-length anti-VEGF protein therapeutic used for treating wet AMD and cancers, was encapsulated inside PDA during the polymerization of dopamine. Stimulated by exogenous oxidative stress, the PDA nanoparticles could achieve the on-demand release of bevacizumab and ROS scavenging simultaneously, which could effectively halt angiogenesis via both anti-VEGF and anti-oxidant pathways and pave a new road for treating diseases related to abnormal angiogenesis.³¹

### Experimental

### Materials

Dopamine hydrochloride and fluorescein isothiocyanate conjugate (FITC)-bovine serum album (BSA) were obtained from Sigma-Aldrich (St. Louis, MO). Bevacizumab (Avastin) was purchased from Genentech, Inc. (San Francisco, CA). Colorimetric (3-(4,5-dimethylthiazol-2-yl)-5-(3-carboxymethoxyphenyl)-2-(4-sulfophenyl)-2H-tetrazolium) (MTS) assay, 2′,7′-dichlorodihydrofluorescein diacetate (DCFH-DA), and lipopolysaccharide (LPS) were purchased from Fisher Scientific Inc. (Hampton, NH). Rhodamine phalloidin, 4′,6-diamidino-2-phenylindole (DAPI), and Amplex red hydrogen peroxide/peroxidase assay kit were purchased from Thermo Fisher Scientific Inc. (Columbus, OH). Human retinal pigment epithelial cells (ARPE-19 cells, CRL2302) were purchased from American Type Culture Collection (Rockville, MD). Human umbilical vein endothelial cells (HUVECs, C0035C), Geltrex LDEV-free reduced growth factor basement membrane matrix, and Invitrogen human VEGF ELISA kit were purchased from Thermo Fisher Scientific Inc. (Waltham, MA). All other reagents used in this study were analytical grade.

### Synthesis of Drug-Loaded PDA Nanoparticles

PDA nanoparticles and drug-loaded PDA nanoparticles were prepared based on methods reported in the literature with some modifications.^{24, 31} Briefly, 100 mg dopamine hydrochloride was added to 50 mL deionized (DI) water containing 420 μL 1N sodium hydroxide solution, which was stirred at 500 rpm for 3-hour polymerization. FITC-BSA was used as a model drug for the particle characterization and the release study of PDA nanoparticles, and bevacizumab, the commonly used anti-VEGF medication for treating wet AMD, was loaded afterward. 100 mg FITC-BSA or bevacizumab was then added to the PDA solution, which was stirred for another 21 hours. Later, the prepared nanoparticles were collected by centrifuge and washed five times with DI water until all the proteins on the surface of the nanoparticles were removed. Then, the PDA nanoparticles were lyophilized and stored at 4° C. until further use.

### Characterization

The morphological characterization of PDA nanoparticles was done by transmission electron microscopy (TEM) (FEI Tecnai G2 Biotwin), and the diameter was determined by measuring at least 100 nanoparticles from TEM images using Image J (NIH). The Zetasizer (Malvern Nano ZS) was also used to determine the hydrodynamic diameter of PDA nanoparticles and drug-loaded nanoparticles. The absorption of PDA nanoparticles at different concentrations was determined by UV-Vis spectrophotometer (Agilent Cary 100).

### Degradation

The degradation of PDA nanoparticles was determined by mixing 200 μg/mL PDA nanoparticles with 10 mM hydrogen peroxide (H₂O₂) diluted in phosphate-buffered saline (PBS) at 37° C. At specific time points, the absorption of degraded PDA nanoparticles was assessed by a UV-Vis spectrophotometer, and their morphological properties were examined by TEM.

### Drug Release and Loading of PDA Nanoparticles

For the oxidative response release, the 1 mg/mL FITC-BSA loaded PDA nanoparticles were dispersed in PBS with 10 mM, 5 mM, 2 mM, 1 mM, and 0 mM (control group) H₂O₂ and incubated at 37° C.³²⁻³⁴ At specific time points, the eluent was collected, and 1 mL of fresh PBS containing different amounts of H₂O₂ was added to the PDA nanoparticles. The eluted FITC-BSA in the supernatant was assessed by a multi-mode microplate reader (BioTek Synergy HTX) (Eₑₓ=485 nm, Eₑₘ=525 nm). Similarly, the release of bevacizumab triggered by different concentrations of H₂O₂ was also studied. The active bevacizumab released from PDA nanoparticles triggered by 10 mM and 0 mM H₂O₂ was assessed by enzyme-linked immunosorbent assay (ELISA).³⁵⁻³⁷ These concentrations were selected to evaluate maximum and minimum release. All the experiments were done in triplicate. The drug loading rate of PDA nanoparticles was determined by dissolving the FITC-BSA or bevacizumab loaded nanoparticles in H₂O₂ and its loading rate was calculated as the total amount of FITC-BSA or bevacizumab loaded into the particles/the mass weight of PDA nanoparticles*100%.

### Cytotoxicity

Cell viability in the presence of PDA nanoparticles was conducted with human retinal pigmented epithelial (ARPE-19) cells.^{37, 38} To be specific, the ARPE-19 cells were seeded in 48-well plates at a density of 4×10⁴ cells/well. The cells were incubated with 0 μg/mL, 1 μg/mL, 2 μg/mL, 5 μg/mL, 10 μg/mL, 20 μg/mL, 50 μg/mL, 100 μg/mL, 200 μg/mL, 500 μg/mL, 1000 μg/mL of PDA nanoparticles for another 24 hours. Then, the particles were removed and cells were treated by MTS reagent for 3 hours at 37° C. The absorbance measurement at 490 nm was obtained by using the microplate reader and normalized to the blank group. The cells were additionally stained by live/dead cell assay and observed by fluorescent microscopy (Nikon, Eclipse TS100) to further determine the influence of PDA nanoparticles on cell viability.³⁹ All experiments were repeated three times, and the result was displayed as average±standard deviation.

### Cellular Uptake of PDA Nanoparticles

Considering the cell viability of PDA nanoparticles, cellular uptake was determined by treating the ARPE-19 cells with 10 μg/mL PDA nanoparticles.⁴⁰ To better visualize the nanoparticles, the PDA nanoparticles were loaded with FITC-BSA. After 24-hour incubation, the cells were stained by 4′,6-diamidino-2-phenylindole (DAPI) and rhodamine-phalloidin and observed under a confocal fluorescence microscope (Zeiss Axio Observer Z1).

### ROS Scavenging by PDA Nanoparticles

To investigate the capability of nanoparticles in scavenging ROS, Amplex red assay was utilized to assess the consumption rate of H₂O₂ by PDA nanoparticles. Briefly, 1 mg blank PDA nanoparticles were treated with 10 mM H₂O₂ and incubated at 37° C. At 30 min, 1 h, 2 h, 4 h, 8 h, 1 day, 2 days, 4 days, and 1 week, the 1 μL of the solution was retrieved and diluted in PBS which was then quantified by using the Amplex red kit. To further confirm the intracellular ROS scavenging capability of PDA nanoparticles, H₂O₂ and LPS (lipopolysaccharide) were used to introduce inflammation using the retinal pigment cell line.^{24, 29} Briefly, ARPE-19 cells were seeded in 24-well plates and 96-well plates at a density of 2×10⁵ cells/mL 24 hours before the study. Then, 200 μM H₂O₂ or 50 μg/mL LPS with 0 μg/mL, 1 μg/mL, 2 μg/mL, 5 μg/mL, 10 μg/mL, 20 μg/mL, or 50 μg/mL PDA nanoparticles were added to the cell and incubated for 24 hours. Afterward, the cells were thoroughly washed with fresh media and treated with 10 μM DCFH-DA for 1 hour.⁴¹ Cells were visualized by a fluorescent microscope, and the intracellular ROS levels revealed by the fluorescent intensity were assessed by a microplate reader and normalized to that of the control group (100%). Each experiment was done in triplicate. The result was displayed as an average±standard deviation.

### VEGF Inhibition Effect of PDA Nanoparticles

To assess the effect of PDA nanoparticles on reducing angiogenesis, a human VEGF ELISA kit was used to quantify the amount of VEGF released from the cells induced by H₂O₂.⁴² Briefly, ARPE-19 cells were seeded in 24-well plates for 24 hours. Then, the cells were incubated with blank media (control group), 200 mM H₂O₂, 200 mM H₂O₂ and 10 μg/mL PDA nanoparticles, 200 mM H₂O₂ and 10 μg/mL bevacizumab loaded nanoparticles, as well as 200 mM H₂O₂ and 5 μg/mL free bevacizumab. After 24 hours, the active VEGF in the media was determined by ELISA. The tube formation assay is a rapid method to assess the angiogenesis of HUVECs induced by ROS.^{43, 44} Under oxidative stress, ARPE-19 cells were co-cultured with HUVECs to investigate the influence of H₂O₂ and drug-loaded PDA nanoparticles on the development of the three-dimensional capillary structure. Briefly, ARPE-19 cells at a concentration of 2×10⁵ cells/mL were seeded on the 12 trans-well plate insert 24 hours before the experiment. Afterward, intracellular inflammation was induced by 200 μM H₂O₂. 10 μg/mL PDA nanoparticles, 10 μg/mL bevacizumab loaded nanoparticles, and 5 μg/mL bevacizumab were also added to the cells to inhibit the production of VEGF, respectively. After 12 hours, the well plate was coated with the basement membrane matrix allowing for the growth of HUVECs.⁴⁴ Then, 800 μL of HUVECs stained by Calcein AM were added to each well and incubated for another 12 hours. The tube formation induced by VEGF secreted from ARPE-19 cells and H₂O₂ was observed by a fluorescent microscope and the tube length was analyzed using Image J. The tube lengths of experimental groups were normalized to the control group (100%). Each condition was done in triplicate. The result was shown as the average±standard deviation.

### Intravitreal Injection of PDA Nanoparticles

To assess the injection feasibility of drug-loaded PDA nanoparticles via a 31-gauge needle, fresh porcine eyes were used as an ex vivo model to mimic clinical intravitreal injection.³⁷ The excess extra orbital tissues were removed from the porcine eyes received from the local abattoir (Bay Packing, Pleasantville, Ohio). Afterward, 100 μL of 200 μg/mL PDA nanoparticles with bevacizumab were loaded into a 1 mL syringe connected to a 31-gauge needle, which is commonly used in the clinical intravitreal injection of bevacizumab for treating wet AMD. The intravitreal injection was placed and the porcine eyes were then bisected. The vitreous humor was collected to assess the injection of PDA nanoparticles. The experiment was done in triplicate.

### Statistical Analysis

Statistical analysis was performed by one-way ANOVA with post-hoc Tukey test by OriginLab. The significance was p<0.05.

### Results and Discussion

### Synthesis of PDA Nanoparticles as a Robust Protein Carrier

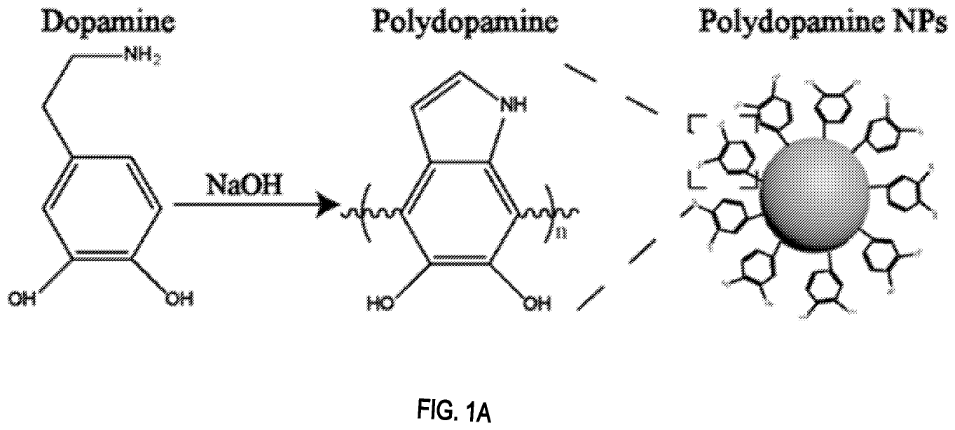

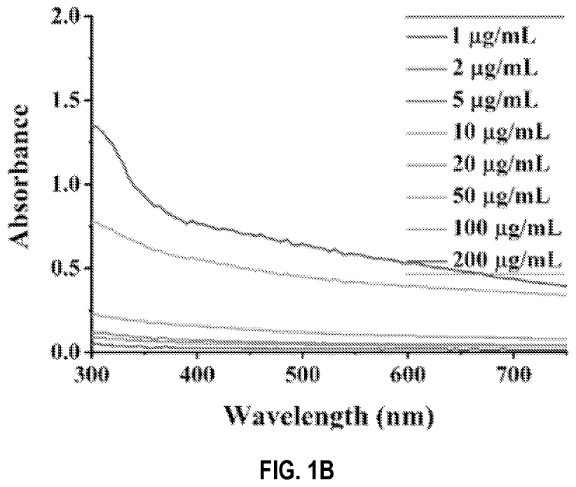

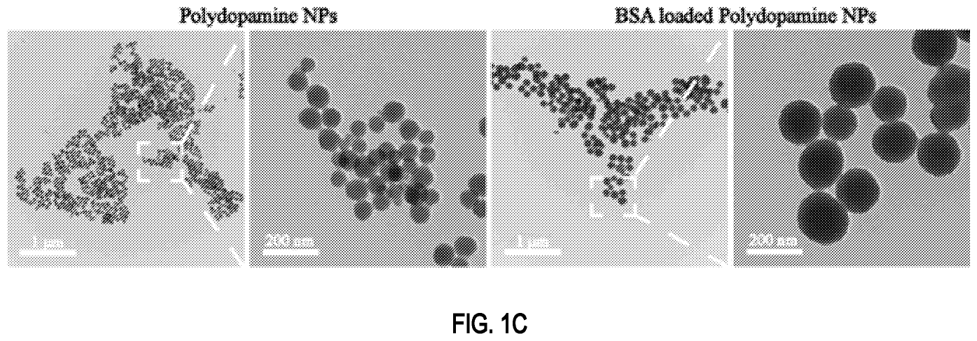

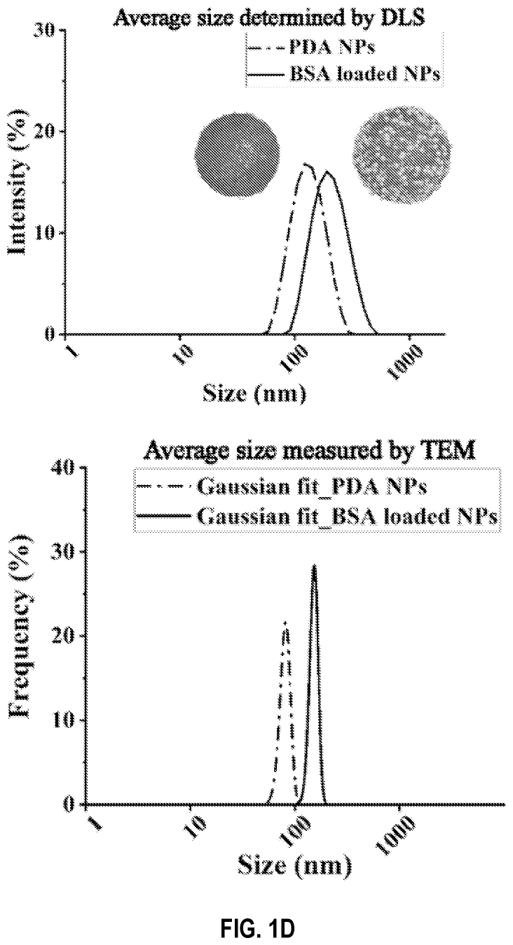

PDA has demonstrated its distinctive functions as a ROS scavenger and drug delivery carrier in previous studies.^{22-24, 25} Several synthetic methods have been developed to prepare PDA nanoparticles, including electro-polymerization, enzymatic polymerization, and chemical oxidation.⁴⁶ The oxidation of dopamine is commonly used due to simple synthesis without sophisticated instruments and harsh reaction conditions. Even though PDA nanoparticles have been widely investigated as vehicles for doxorubicin delivery, its capability as a carrier for targeted delivery of protein therapeutics remains mostly unknown. As such, PDA nanoparticles were synthesized via solution-oxidation method and protein therapeutics loaded into the PDA nanoparticles by physically entrapping the drug inside the nanoparticle while the polymeric chains were growing. Under alkaline conditions, the polymerization of dopamine immediately occurred, indicated by the solution color which turned brownish to dark black.⁴⁷ As shown in FIGS. 1A-1D, the successful synthesis of PDA nanoparticles was confirmed by UV-Vis absorption spectroscopy, TEM, and dynamic light scattering (DLS). The characteristic TEM images reveal the successful preparation of nanoparticles.

It has been known that nanoparticles with sizes ranges of 15-240 nm and spherical shapes demonstrate good cellular uptake efficiency.⁴⁸ The nanoparticles prepared were found to be in this preferred range with a spherical shape and an average diameter of 81.62±9.20 nm, characterized by TEM and DLS. Additionally, uniformity of nanoparticles is critical for achieving consistency in cell studies and drug delivery applications. The uniform size of PDA nanoparticles was confirmed by calculating the polydispersity of the nanoparticles, which is approximately 0.11. Taken together, an oxidation-based approach for synthesizing PDA nanoparticle within a proper shape, size, and high uniformity for drug delivery applications was established.

Next, the capability of PDA nanoparticles for efficient protein loading was investigated, which is required for long-term protein delivery applications. In most polymer-based nanoparticles, their interactions with drugs are based upon hydrophobic interactions, hydrogen bonds, and physisorption. In contrast, PDA nanoparticles, with abundant n electrons from the aromatic rings and quinones, can provide additional n-n interactions to interact with aromatic amino acids. As such, it was hypothesized that PDA nanoparticles could achieve a high loading capacity. As a proof-of-concept, BSA protein containing histidine, phenylalanine, tryptophan, which is similar to bevacizumab, was used as a model drug to optimize the experimental conditions, the successful physical binding of BSA to the PDA nanoparticles was confirmed first by incubating PDA nanoparticles with free BSA, followed by the collection of particles. The physical encapsulation of protein by PDA nanoparticles was proved by TEM, where a significant increase in the size of PDA nanoparticles (152.99±14.09 nm) was observed after incubation with BSA protein compared to free PDA nanoparticles (81.62±9.20 nm) (p<0.05). Later, the high drug loading capacity of PDA nanoparticles was confirmed by dissolving a certain amount of BSA loaded PDA nanoparticles in the H₂O₂ solution. However, the black color of the PDA nanoparticles may influence the quantification of BSA based on absorbance. Therefore, the drug loading capacity was assessed by using fluorescent protein, FITC-BSA, to facilitate the precise quantification of protein concentrations. By dissolving the FITC-BSA loaded nanoparticles, a loading rate of 40.84±1.91% was found by normalizing the total amount of encapsulated BSA to the mass of total nanoparticles, which represents an advancement compared to most reported devices.⁴⁹ In short, the synthesized PDA nanoparticle system enables efficient protein loading and has proper sizes and shapes for drug delivery applications.

### Cytotoxicity of PDA Nanoparticles

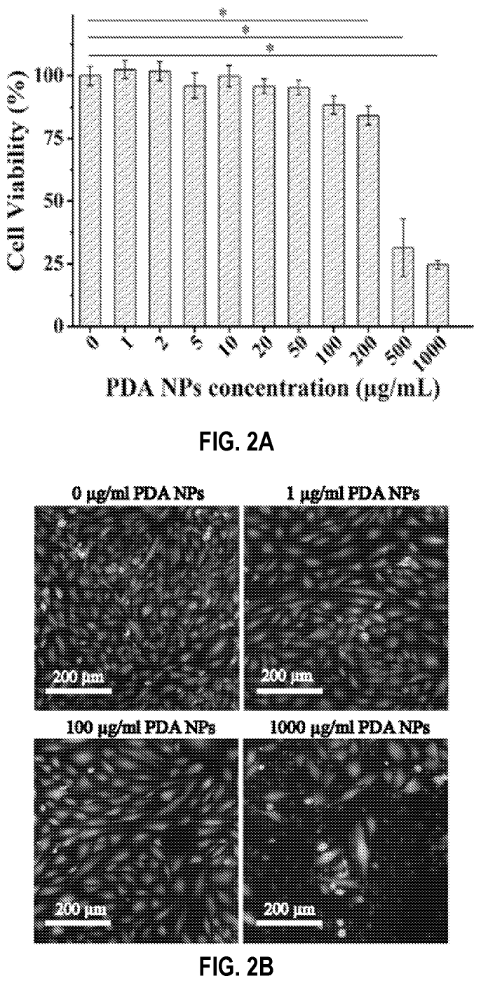

One of the most fundamental criteria for developing a drug delivery system is biocompatibility.⁵⁰ As such, after the synthesis of PDA nanoparticles, their biocompatibility was studied using a human retinal cell line. Dopamine naturally exists in the human brain, and several studies have shown low cytotoxicity from PDA nanoparticles in human fibroblast and neuronal cell lines.⁵¹ Therefore, good ocular biocompatibility of PDA nanoparticles was hypothesized and subsequently verified utilizing human retinal pigment epithelial (RPE) cells. Specifically, we incubated ARPE-19 cells with PDA nanoparticles at different concentrations for 24 hours, then assessed the cell metabolic activity by MTS assay and live/dead assay. As shown in FIGS. 2A-2B, high concentrations of PDA nanoparticles might lead to cell apoptosis in 24 hours. Approximately 25% of the cells survived after one-day co-culture with 1000 μg/mL PDA nanoparticles. However, the PDA nanoparticles with a concentration below 100 μg/mL did not show an apparent cytotoxic effect, which was also demonstrated by the live/dead assay. The cell survival rate was maintained above 85% when in contact with a proper amount of PDA nanoparticles below 100 μg/mL. Therefore, these results revealed the acceptable biocompatibility of PDA nanoparticles on ocular cells in vitro.

### Tunable and ROS-Responsive Biodegradation of PDA Nanoparticles

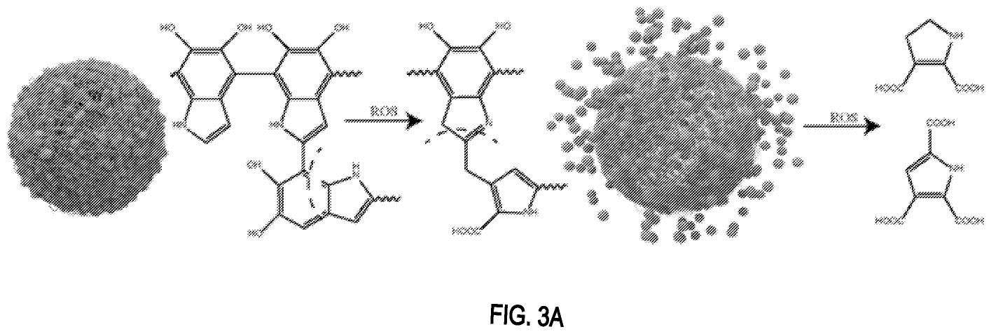

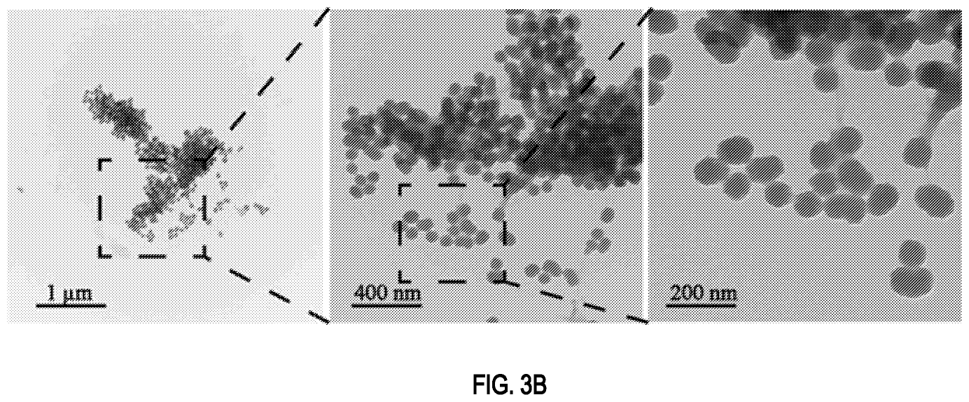

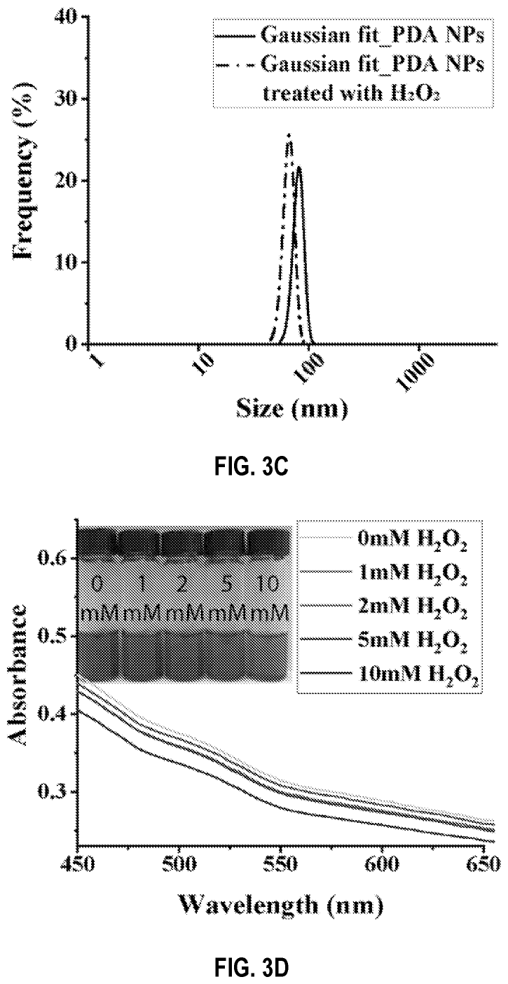

In addition to biocompatibility, proper biodegradability is also essential for drug delivery applications. It was hypothesized that our PDA nanoparticle-based drug delivery system is biodegradable through ROS based on a redox reaction. The most common ROS include H₂O₂, HO^{•} radicals, and O^{•-} radicals.⁵² Among them, H₂O₂ concentration has been associated with the greatest degree of damage after AMD development.^{53, 54} Therefore, the modulation of ROS-triggered degradation of PDA nanoparticles was demonstrated by varying the concentrations of H₂O₂. To accelerate the biodegradation of nanoparticles, the concentrations of H₂O₂ solution used for treating PDA nanoparticles were above 1 mM. TEM and UV-Vis absorption spectroscopy were utilized to characterize the biodegradation of PDA nanoparticles. Specifically, after incubation for one week, the edges of the nanoparticles became blurred and the particles lost its major part as shown in TEM images in FIGS. 3A-3D. The size of PDA nanoparticles significantly reduced to 66.69±7.78 nm from the original size of 81.62±9.20 nm, on average (p<0.05). The diameter of PDA nanoparticles was decreased by 18.3% after one-week incubation with 10 mM H₂O₂ treatment. Additionally, the absorption of PDA nanoparticles decreased as the amount of H₂O₂ increased, and its color faded over time, which strongly suggested the degradation of PDA nanoparticles. The biodegradation of PDA happened in the presence of oxidative species attacking the nucleophiles carbonyl and formed pyrrole-2,3-dicarboxylic acid (PTCA) and pyrrole-2,3-dicarboxylic acid (PDCA) oligomers via redox reaction.^{21, 55, 56} It was also found that the biodegradation products contain dopamine, PDA segments, and quinine, and these byproducts could further react with ROS and reduce the level of oxidative stress.⁵⁷ As such, the PDA nanoparticles are biodegradable under physiologically relevant conditions, demonstrating potential suitability for further in vivo validation.

### Intracellular ROS Scavenging by PDA Nanoparticles

After demonstrating their in vitro biocompatibility and biodegradability, the effective scavenging of ROS utilizing PDA nanoparticles was demonstrated. It was hypothesized that the PDA nanoparticles have the ability to scavenge ROS, as ROS is consumed during the biodegradation of PDA nanoparticles. Herein, the Amplex red was firstly used to confirm the H₂O₂ consumption rate by PDA nanoparticles. By treating the particles with H₂O₂ for one week, the concentration of H₂O₂ concentration in the solution dropped to 6.69±0.77 mM from the initial concentration of 10 mM during the first 24 hours because the H₂O₂ reacted with PDA nanoparticles. The depletion rate of H₂O₂ was calculated as approximately 0.14 mM per hour. However, the rate of H₂O₂ consumption decreased to 0.02 mM per hour after one day, likely because most particles were deposited at the bottom of the solution, which reduced the contact and reaction rate between H₂O₂ and PDA nanoparticles. Overall, PDA nanoparticles can steadily react with H₂O₂ and reduce oxidative stress over time.

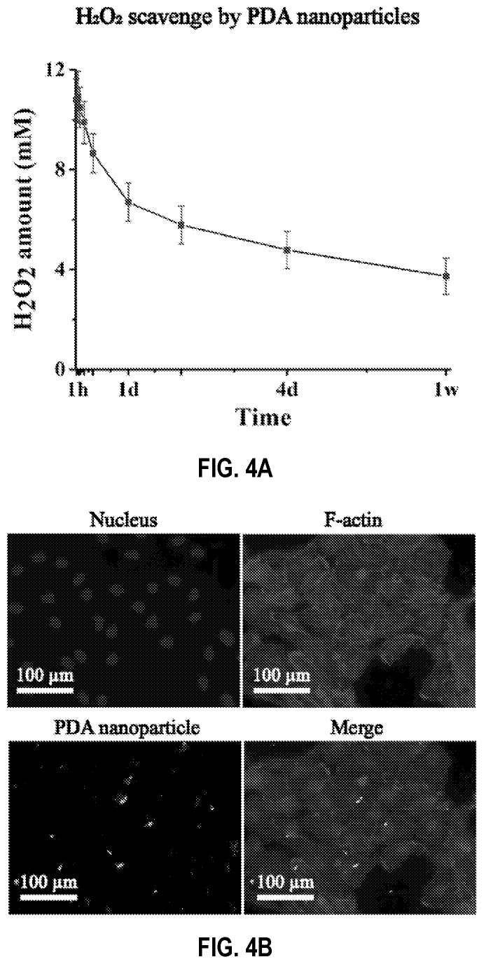

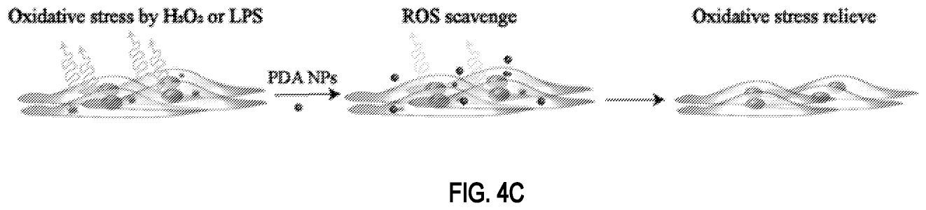

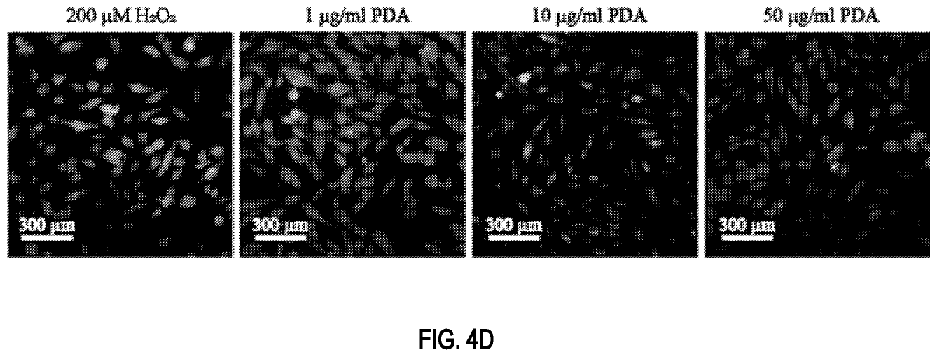

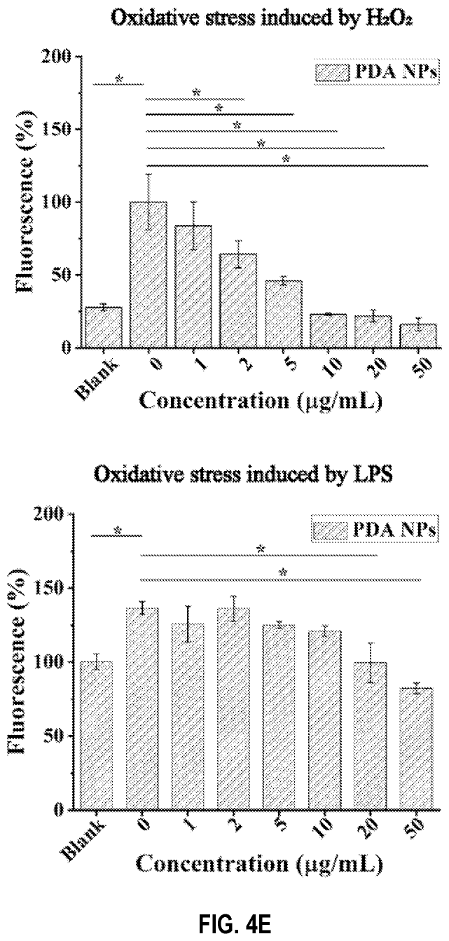

The effective reduction of intracellular ROS has been considered as an effective strategy to treat AMD, as previous studies have shown that accumulation of intracellular ROS in retinal cells can lead to angiogenesis.¹¹ First, the high cellular uptake efficiency of PDA nanoparticles was confirmed, as the generation of ROS always emerges and remains in the cytoplasm. Specifically, the cellular distribution of nanoparticles was studied by treating APRE-19 cells with FITC-BSA loaded PDA nanoparticles, which can be monitored by their bright green fluorescence. As shown in FIG. 4A, favored by the small size of the nanoparticles, most nanoparticles were trafficked into the cells and located in the cytoplasm, which could enhance intracellular ROS scavenging. Moreover, the small size of the nanoparticles may potentially improve ROS depletion due to higher surface area compared to larger microparticles.

To demonstrate the capability of PDA nanoparticles for scavenging ROS intracellularly, ARPE-19 cells were first treated by H₂O₂ to induce a high level of ROS and mimic the diseased microenvironment seen in AMD. The successful induction of ROS level was then characterized by the increased DCFH-DA green fluorescence compared to the control (no treatment). However, an obvious decrease in fluorescent intensity was observed with an increasing concentration of PDA nanoparticles. It was speculated that the redox reaction between PDA and H₂O₂ alleviated the oxidative stress on the cells. Beyond that, PDA could also act as a catalyzer consuming the excess H₂O₂ in the media by decomposing the H₂O₂ into oxygen, potentially increasing the survival rate of cells.²⁴ As the high level of ROS during the progression of AMD could also be triggered by inflammation, in this study, RPE cells were also treated by bacteria LPS which could also lead to strong oxidative stress.^{57, 58} The upregulated ROS levels were confirmed by DCFH-DA fluorescence. Strikingly, the ROS level decreased significantly after adding PDA nanoparticles (p<0.05), as previous reports showed.¹⁷ Mechanistically, the PDA nanoparticles could inhibit ROS production. These results suggest the potential of using these PDA nanoparticles as ROS scavengers for the treatment of AMD.

### Drug Loading and ROS Responsive Release of PDA Nanoparticles

While PDA nanoparticle-based scavenging of ROS may provide neuroprotective effects in AMD, anti-VEGF delivery could continue to take immediate action on the angiogenesis and slow down the progression of AMD. The efficient loading and controlled releasing of protein therapeutics was next investigated, considering the effective treatment of AMD requires a prolonged release of anti-VEGF. The drug loading rate is closely related to the performance of nanomedicine release.⁵⁹ The high drug loading content could meet the high dosage requirement for long-term administration and further extend the intravitreal injection cycles to improve patients' compliance. After confirming the FITC-BSA loading of PDA nanoparticles, bevacizumab, the clinical therapeutics for treating wet AMD, was loaded to the PDA nanoparticles. Approximately 341.52 μg bevacizumab was entrapped in 1 mg nanoparticles, and the loading rate was 34.15±3.31%. The loading rate of bevacizumab was lower than that of FITC-BSA. The difference in loading rates might be caused by the higher molecular weight of bevacizumab, resulting in difficulties in encapsulating the larger protein in the small cavities of PDA nanoparticles. However, the bevacizumab loading rate of PDA nanoparticles is still higher than the most reported devices for treating wet AMD, indicating its potential in preserving higher dosage of protein therapeutics and extend the release period up to several months as a protein drug carrier.⁶⁰

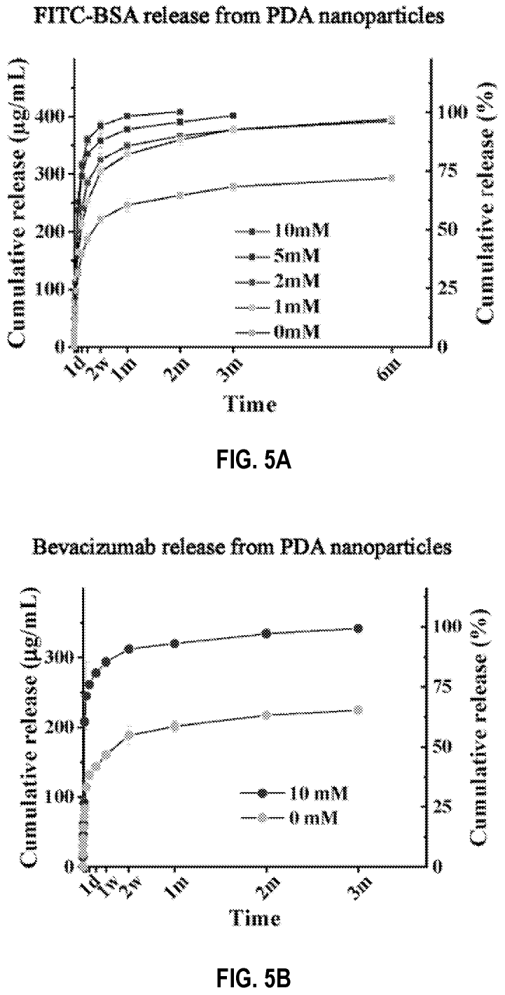

As aforementioned, the ROS could trigger the degradation of PDA nanoparticles and simultaneously accelerate the release of drug encapsulated inside the particles. Therefore, the drug release rate of nanoparticles could be controlled and tailored by ROS accumulated intraocularly, and the on-demand release of protein therapeutics could be achieved. To clearly investigate the relationship between the level of ROS and release rate, a higher concentration of H₂O₂, as the oxidative agent inducing the degradation of PDA nanoparticles and stimulating the release of FITC-BSA, in the range of 1 mM to 10 mM was used in the study as compared to the moderate concentration of 100-300 μM H₂O₂ commonly used to conduct the in vitro cellular inflammation and 25-70 μM typical ROS level in humans.⁶¹ As shown in FIG. 5A, the two phases of protein release with an initial burst release triggered by surface erosion and diffusion of the proteins adsorbed to the surface, followed by a more sustained release from the proteins trapped inside the nanoparticles was observed. The initial diffusion of PDA chains that are not crosslinked to the nanoparticle can contribute to an initial decrease in size. Moreover, the quick-release of FITC-BSA at the beginning caused by a faster decrease in size triggered by ROS was found. The higher concentration of ROS could lead to a faster release of FITC-BSA. Specifically, more than 85% of FITC-BSA was eluted from the nanoparticles when adding 10 mM H₂O₂ in the first two weeks. However, only around 55% of FITC-BSA was detected in the control group without any H₂O₂ during the same period. PDA nanoparticles presented a moderate release rate when treated with 5 mM, 2 mM, or 1 mM H₂O₂, suggesting that the oxidative species could significantly improve the drug release rate by accelerating the degrading process of PDA nanoparticle. However, after two weeks, most of the uncrosslinked segments of nanoparticles have already diffused from the system, and the concentration of H₂O₂ could also rapidly decrease. A phase of steady release was then seen at later time points. PDA nanoparticles treated by 10 mM H₂O₂ exhibited a slow release rate that dropped to 0.58 μg/mL per day limited by the significant loss of FITC-BSA in the first two weeks. After two months, all the particles under 10 mM H₂O₂ degraded and all the encapsulated FITC-BSA was released. Similarly, the PDA nanoparticles treated with 5 mM H₂O₂ completely the degraded and released all protein by three months. On the contrary, the release rate of the PDA nanoparticles treated with 2 mM, 1 mM, and 0 mM (control group) maintained release at approximately 0.40-0.50 μg/mL per day between two weeks and six months, in a sustained release manner, which indicated the possibility of PDA nanoparticles to release out to six months. Moreover, it was still observed that the PDA nanoparticles did not fully degrade after six months under the lower levels of oxidative stress, indicating that the PDA nanoparticle may sustainably release the protein therapeutics in the physiological microenvironment with relatively low ROS concentration of approximately 25-70 μM, which could still trigger release. Accordingly, the PDA nanoparticle demonstrates excellent potential for the long-term sustained release of protein therapeutics and on-demand release of protein therapeutics under oxidative stress.

For treating AMD, monoclonal full-length anti-VEGF, bevacizumab, is the most commonly used medication. As such, the capability of PDA nanoparticles on releasing bevacizumab led by two extreme concentrations of ROS (10 mM and 0 mM H₂O₂) was also assessed by ELISA. The general trend of H₂O₂ triggered release was summarized in FIG. 5B. Similar to the release profile of FITC-BSA, an initial burst release of bevacizumab was also found, and higher ROS led to a quicker release of protein therapeutics. The group with 10 mM H₂O₂ presented the highest release rate, and more than 300 μg active bevacizumab was released from the nanoparticles during the first two weeks. However, the control group without any H₂O₂ treatment only released 188.30±13.34 μg bevacizumab during the same period, which revealed the positive effect of ROS on triggering drug release. Between two weeks and three months, the release rates of the groups treated by 10 mM and 0 mM H₂O₂ were both stabilized at around 0.40-0.50 μg per day. The degradation of bevacizumab loaded nanoparticles treated with 10 mM H₂O₂ was completed at three months, which was slightly longer than that of FITC-BSA loaded nanoparticles. As expected, bevacizumab loaded nanoparticles without any H₂O₂ treatment could sustainably release bevacizumab and did not completely degrade after three months. Taken together, the sustained drug release and triggered drug release can co-exist in this PDA nanoparticle-based system under relatively low levels of oxidative stress. The release of bevacizumab could be regulated by the oxidative species by reacting with the nanoparticles, which have the potential to further inhibit the development of angiogenesis via both anti-VEGF and antioxidant pathways. However, future in vivo studies are still essential in further investigating the pharmacokinetics of PDA nanoparticles releasing anti-VEGF in the physiological environment to prove the effectiveness of ROS-responsive sustained release of anti-VEGF.

### Effect of PDA Nanoparticles on VEGF Production and Tube Formation

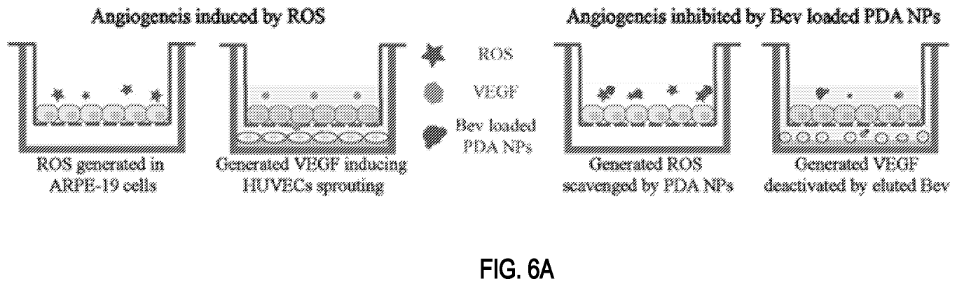

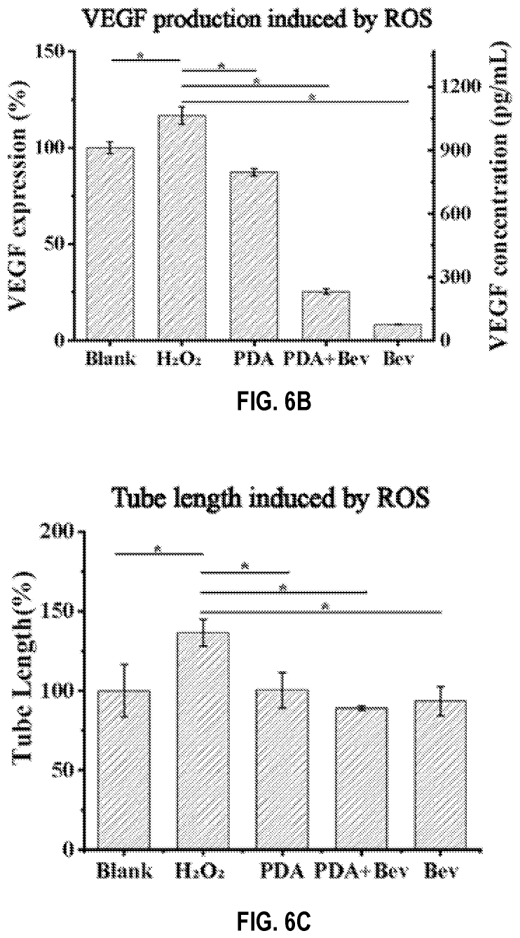

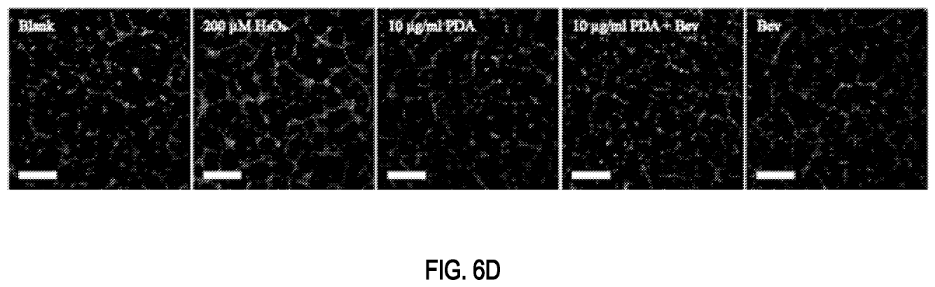

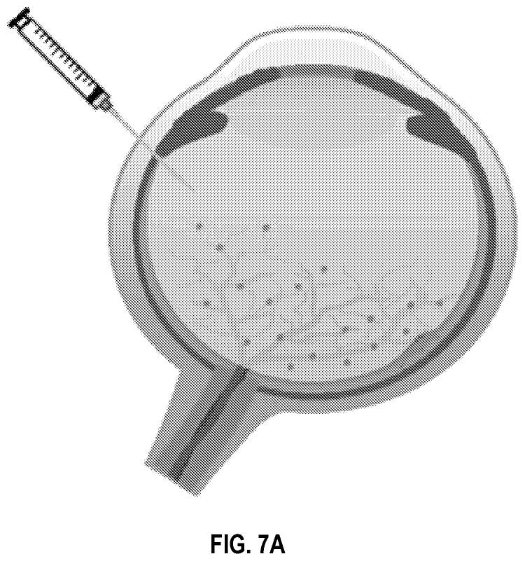

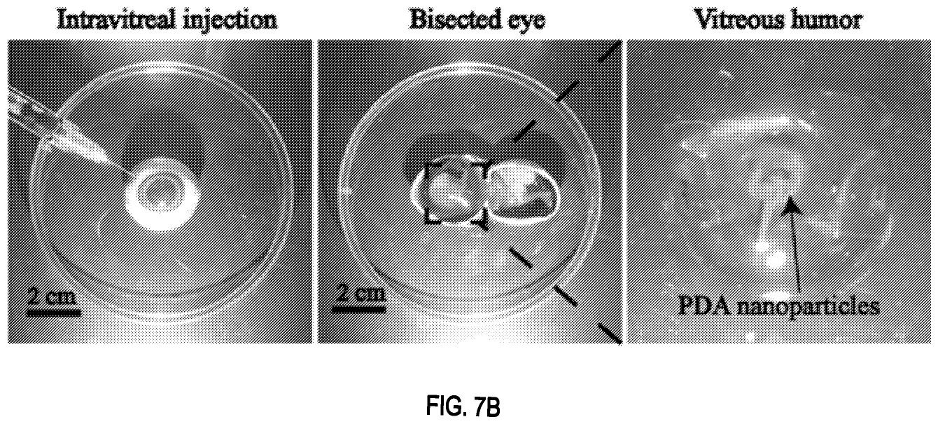

ROS is the critical cellular mediator, which could further stimulate the production of VEGF and regulate angiogenesis. By eliminating the ROS, the generation of VEGF and the development of angiogenesis could theoretically be controlled. To investigate the capability of PDA nanoparticles and bevacizumab loaded nanoparticles on reducing the production of VEGF, ELISA was used to quantify the amount of VEGF secreted by ARPE-19 cells stimulated by H₂O₂. As shown in FIGS. 6A-6D, the cells under the oxidative stress produced 116.70±4.41% VEGF as compared to the control group. However, this number significantly reduced to 87.31±1.99% when adding PDA nanoparticles to diminish the spare H₂O₂, as shown in FIG. 6B (p<0.05). Moreover, the amount of free active VEGF significantly dropped to 25.31±1.45% and 8.27±0.14% respectively of the group with 10 μg/mL bevacizumab loaded PDA nanoparticles and 5 μg/mL native bevacizumab (p<0.05). As such, the PDA nanoparticles could effectively scavenge the added H₂O₂ and reduce VEGF production. Moreover, more active VEGF could be blocked by the bevacizumab loaded PDA nanoparticle, suggesting its potential in systematic AMD treatment by interfering with the level of ROS and VEGF.

Even though VEGF ELISA could reveal the inhibition effect of bevacizumab loaded PDA nanoparticles on VEGF generation, the influence of PDA nanoparticles on ROS directing angiogenesis via independent VEGF pathway still needs investigation by endothelial cell tube formation assay. The tube formation assay is a reliable method to quantify the most angiogenic stimuli.⁴⁴ Considering the CNV is also involving the VEGF-independent pathway, this assay could assess the inhibition effect of particles on various angiogenesis factors inducing tube development. The significantly improved anti-angiogenesis was confirmed based on the tube-formation assay utilizing the combined therapy from ROS scavenging by PDA nanoparticles and the simultaneous delivery of anti-VEGF. Specifically, in the presence of ROS, H₂O₂, the capillary-like structure was formed, which indicated that more VEGF and other angiogenetic factors were produced to trigger the HUVEC tube formation as compared to the control group without any H₂O₂. A significant decrease in tube length was observed in the group treated by PDA nanoparticles which reacted to the added H₂O₂ and reduced the oxidative stress. The PDA nanoparticle group has a similar tube length with the blank group, indicating the PDA nanoparticle could alleviate the stress. For the group treated by bevacizumab loaded nanoparticles, HUVECs cells were well dispersed and did not sprout out obviously because the released bevacizumab could further effectively disturb the tube formation and inhibit angiogenesis development. As a result, the tube length decreased to 89.14±1.28% from 100.37±11.15% significantly (p<0.05). However, it is interesting to find that the native bevacizumab had a similar length which is 93.58±9.27% as compared to the group treated by drug-loaded particles. Based on the ELISA of VEGF, a lower amount of active VEGF was found in the group with native free bevacizumab. It is probably caused by PDA that could inhibit the tube formation via the VEGF-independent pathway. Therefore, the differences between the group of native bevacizumab and bevacizumab loaded nanoparticle on tube length were not significant. Taken together, the bevacizumab loaded PDA nanoparticles could synergistically prevent the new blood vessel formation by downregulating VEGF signaling pathway and VEGF-independent pathway.

### Intravitreal Injection of PDA Nanoparticles

Intravitreal injection is currently the most effective procedure for treating AMD that directly delivers anti-VEGF to the retina as compared to other administration approaches. For the current standard of treating AMD, a 31- or 32-gauge needle is applied in the intravitreal injection to avoid tissue trauma, minimize pain from the injection, improving patients' acceptance.⁶² However, these small gauge needles are currently only used in delivering soluble agents. As aforementioned, the PDA nanoparticles could be well dispersed in aqueous solutions, favoring injection. Moreover, the particles were designed smaller than 200 nm by controlling the pH during particle preparation, so the retention of nanoparticles in a 31-gauge needle could be avoided.⁶³

To perform the intravitreal injection and study the injection feasibility of bevacizumab loaded PDA nanoparticles, fresh porcine eyes were used as an ex vivo model and received the intravitreal injection of 100 μL nanoparticles dispersed in PBS via a 31-gauge needle. After injection, the nanoparticles were not obviously retained in the syringe or syringe needle due to its high water dispersibility and small size. After dissection of the porcine eye, the black PDA nanoparticles were located in the vitreous humor. Also, both blank nanoparticles and drug-loaded nanoparticles were negatively charged, as shown in Table 1. The favorable size and surface charge may assist with the free movement of the particles to the target region: the deep retinal layer. The distribution and movement of PDA nanoparticles in both the vitreous humor and retina will be fully investigated in the future using in vivo models. Overall, the PDA nanoparticles could be delivered to the eye via a 31-gauge needle.

TABLE 1
Summarized loading rate and zeta potential of PDA
nanoparticles, FITC-BSA loaded nanoparticles
and bevacizumab loaded nanoparticles.

| | | FITC-BSA | Bevacizumab |
|---|---|---|---|
| | PDA | loaded | loaded |
| | nanoparticles | nanoparticles | nanoparticles |
| Loading capacity | / | 40.84 ± 1.91% | 34.15 ± 3.31% |
| Zeta Potential (mV) | −27.28 ± 0.81 | −14.10 ± 6.34 | −8.19 ± 4.71 |

### Conclusion

In conclusion, the first nanoparticle-based reduction of ROS and delivery of anti-VEGF for the suppression of angiogenesis was successfully established. Their synergistic effects were confirmed through a tube formation assay that mimics the key pathology of neovascularization in AMD. Meanwhile, the ROS-triggered release of anti-VEGF via accelerating biodegradation of nanoparticles also indicates the potential of our platform for on-demand drug release. Given the prevalence of diseases associated with both ROS and vascularization and the unique biochemical properties demonstrated by PDA nanoparticles, the disclosed platform holds great potential for the treatment of AMD and a variety of other tissue diseases and injuries resulting from free radical damage or angiogenesis such as tumor growth. Moving forward, an in vivo evaluation of the biocompatibility, and drug release performance of the nanoparticles would facilitate the understanding of our drug delivery system under more clinically relevant conditions. In parallel, verifying the synergistically enhanced anti-angiogenic efficacy from our PDA nanoparticle-based anti-VEGF delivery platform in a specific disease model (e.g. rabbit AMD model) would also be required before they could be clinically translated to the treatment of AMD as well as other diseases with abnormal angiogenesis.

### REFERENCES

Each of the below references are independently incorporated by referenced herein in their entireties for all purposes.
1. Folkman, J., Angiogenesis. In Biology of endothelial cells, Springer: 1984; pp 412-428.
2. Risau, W., Mechanisms of angiogenesis. Nature 1997, 386 (6626), 671-674.
3. Carmeliet, P.; Jain, R. K., Angiogenesis in cancer and other diseases. nature 2000, 407 (6801), 249-257.
4. Xian, D.; Song, J.; Yang, L.; Xiong, X.; Lai, R.; Zhong, J., Emerging Roles of Redox-Mediated Angiogenesis and Oxidative Stress in Dermatoses. Oxidative Medicine and Cellular Longevity 2019, 2019, 2304018.
5. Jager, R. D.; Mieler, W. F.; Miller, J. W., Age-related macular degeneration. New England Journal of Medicine 2008, 358 (24), 2606-2617.
6. de Jong, P. T. V. M., Age-Related Macular Degeneration. New England Journal of Medicine 2006, 355 (14), 1474-1485.
7. Avery, R. L.; Castellarin, A. A.; Steinle, N. C.; Dhoot, D. S.; Pieramici, D. J.; See, R.; Couvillion, S.; Ma'an, A. N.; Rabena, M. D.; Le, K., Systemic pharmacokinetics following intravitreal injections of ranibizumab, bevacizumab or aflibercept in patients with neovascular AMD. British Journal of Ophthalmology 2014, 98 (12), 1636-1641.
8. Jager, R. D.; Aiello, L. P.; Patel, S. C.; Cunningham Jr, E. T., Risks of intravitreous injection: a comprehensive review. Retina 2004, 24 (5), 676-698.
9. Schieber, M.; Chandel, N. S., ROS function in redox signaling and oxidative stress. Curr Biol 2014, 24 (10), R453-R462.
10. Dong, A.; Xie, B.; Shen, J.; Yoshida, T.; Yokoi, K.; Hackett, S. F.; Campochiaro, P. A., Oxidative stress promotes ocular neovascularization. Journal of cellular physiology 2009, 219 (3), 544-552.
11. Blasiak, J.; Petrovski, G.; Veréb, Z.; Facskó, A.; Kaarniranta, K., Oxidative stress, hypoxia, and autophagy in the neovascular processes of age-related macular degeneration. BioMed research international 2014, 2014.
12. Sapieha, P.; Hamel, D.; Shao, Z.; Rivera, J. C.; Zaniolo, K.; Joyal, J. S.; Chemtob, S., Proliferative retinopathies: Angiogenesis that blinds. The International Journal of Biochemistry & Cell Biology 2010, 42 (1), 5-12.
13. Winkler, B. S.; Boulton, M. E.; Gottsch, J. D.; Sternberg, P., Oxidative damage and age-related macular degeneration. Molecular vision 1999, 5, 32.
14. Beatty, S.; Koh, H.-H.; Phil, M.; Henson, D.; Boulton, M., The role of oxidative stress in the pathogenesis of age-related macular degeneration. Survey of ophthalmology 2000, 45 (2), 115-134.
15. Nowak, J. Z., Age-related macular degeneration (AMD): pathogenesis and therapy. Pharmacological Reports 2006, 58 (3), 353.
16. Khandhadia, S.; Lotery, A., Oxidation and age-related macular degeneration: insights from molecular biology. Expert reviews in molecular medicine 2010, 12.
17. Yasukawa, T.; Ogura, Y.; Sakurai, E.; Tabata, Y.; Kimura, H., Intraocular sustained drug delivery using implantable polymeric devices. Advanced Drug Delivery Reviews 2005, 57 (14), 2033-2046.
18. Thrimawithana, T. R.; Young, S.; Bunt, C. R.; Green, C.; Alany, R. G., Drug delivery to the posterior segment of the eye. Drug Discovery Today 2011, 16 (5), 270-277.
19. Liu, Y.; Ai, K.; Lu, L., Polydopamine and its derivative materials: synthesis and promising applications in energy, environmental, and biomedical fields. Chemical reviews 2014, 114 (9), 5057-5115.
20. Lynge, M. E.; van der Westen, R.; Postma, A.; Städler, B., Polydopamine—a nature-inspired polymer coating for biomedical science. Nanoscale 2011, 3 (12), 4916-4928.
21. Bettinger, C. J.; Bruggeman, J. P.; Misra, A.; Borenstein, J. T.; Langer, R., Biocompatibility of biodegradable semiconducting melanin films for nerve tissue engineering. Biomaterials 2009, 30 (17), 3050-3057.
22. Wang, Q.; Zhang, R.; Lu, M.; You, G.; Wang, Y.; Chen, G.; Zhao, C.; Wang, Z.; Song, X.; Wu, Y., Bioinspired polydopamine-coated hemoglobin as potential oxygen carrier with antioxidant properties. Biomacromolecules 2017, 18 (4), 1333-1341.
23. Bao, X.; Zhao, J.; Sun, J.; Hu, M.; Yang, X., Polydopamine nanoparticles as efficient scavengers for reactive oxygen species in periodontal disease. ACS nano 2018, 12 (9), 8882-8892.
24. Zhao, H.; Zeng, Z.; Liu, L.; Chen, J.; Zhou, H.; Huang, L., Huang, J., Xu, H.; Xu, Y.; Chen, Z., Polydopamine nanoparticles for the treatment of acute inflammation-induced injury. Nanoscale 2018, 10 (15), 6981-6991.
25. Rodriguez, A. R. C.; Saiz-Poseu, J.; Garcia-Pardo, J.; Garcia, B.; Lorenzo, J.; Ojea-Jiménez, I.; Komilis, D.; Sedó, J.; Busqué, F.; Sánchez, A., Biocompatible polydopamine-like particles for the removal of heavy metals at extremely low concentrations. RSC Advances 2016, 6 (46), 40058-40066.
26. Snodderly, D. M., Evidence for protection against age-related macular degeneration by carotenoids and antioxidant vitamins. The American journal of clinical nutrition 1995, 62 (6), 1448S-1461 S.
27. van Leeuwen, R.; Boekhoorn, S.; Vingerling, J. R.; Witteman, J. C. M.; Klaver, C. C. W.; Hofman, A.; de Jong, P. T. V. M., Dietary intake of antioxidants and risk of age-related macular degeneration. Jama 2005, 294 (24), 3101-3107.
28. Cai, X.; McGinnis, J. F., Nanoceria: a potential therapeutic for dry AMD. In Retinal Degenerative Diseases, Springer: 2016; pp 111-118.
29. Zhou, X.; Wong, L. L.; Karakoti, A. S.; Seal, S.; McGinnis, J. F., Nanoceria inhibit the development and promote the regression of pathologic retinal neovascularization in the Vldlr knockout mouse. PloS one 2011, 6 (2), e16733.
30. Charbgoo, F.; Ahmad, M. B.; Darroudi, M., Cerium oxide nanoparticles: green synthesis and biological applications. International journal of nanomedicine 2017, 12, 1401-1413.
31. Dong, Z.; Gong, H.; Gao, M.; Zhu, W.; Sun, X.; Feng, L.; Fu, T.; Li, Y.; Liu, Z., Polydopamine nanoparticles as a versatile molecular loading platform to enable imaging-guided cancer combination therapy. Theranostics 2016, 6 (7), 1031.
32. Napoli, A.; Valentini, M.; Tirelli, N.; Müller, M.; Hubbell, J. A., Oxidation-responsive polymeric vesicles. Nature Materials 2004, 3 (3), 183-189.
33. Cheng, Y.; Jiao, X.; Xu, T.; Wang, W.; Cao, Y.; Wen, Y.; Zhang, X., Free-Blockage Mesoporous Anticancer Nanoparticles Based on ROS-Responsive Wetting Behavior of Nanopores. Small 2017, 13 (40), 1701942.
34. Tao, W.; He, Z., ROS-responsive drug delivery systems for biomedical applications. Asian Journal of Pharmaceutical Sciences 2018, 13 (2), 101-112.
35. Varshochian, R.; Riazi-Esfahani, M.; Jeddi-Tehrani, M.; Mahmoudi, A. R.; Aghazadeh, S.; Mahbod, M.; Movassat, M.; Atyabi, F.; Sabzevari, A.; Dinarvand, R., Albuminated PLGA nanoparticles containing bevacizumab intended for ocular neovascularization treatment. Journal of Biomedical Materials Research Part A 2015, 103 (10), 3148-3156.
36. Jiang, P.; Chaparro, F. J.; Cuddington, C. T.; Palmer, A. F.; Ohr, M. P.; Lannutti, J. J.; Swindle-Reilly, K. E., Injectable biodegradable bi-layered capsule for sustained delivery of bevacizumab in treating wet age-related macular degeneration. Journal of Controlled Release 2020, 320, 442-456.
37. Jiang, P.; Jacobs, K. M.; Ohr, M. P.; Swindle-Reilly, K. E., Chitosan-Polycaprolactone Core-Shell Microparticles for Sustained Delivery of Bevacizumab. Molecular Pharmaceutics 2020.
38. Luis de Redin, I.; Boiero, C.; Martinez-Ohárriz, M. C.; Agüeros, M.; Ramos, R.; Peñuelas, I.; Allemandi, D.; Llabot, J. M.; Irache, J. M., Human serum albumin nanoparticles for ocular delivery of bevacizumab. International Journal of Pharmaceutics 2018, 541 (1), 214-223.
39. Lai, J.-Y.; Li, Y.-T.; Wang, T.-P., In vitro response of retinal pigment epithelial cells exposed to chitosan materials prepared with different cross-linkers. Int J Mol Sci 2010, 11 (12), 5256-5272.
40. Wang, K.; Mitra, R. N.; Zheng, M.; Han, Z., Nanoceria-loaded injectable hydrogels for potential age-related macular degeneration treatment. Journal of Biomedical Materials Research Part A 2018, 106 (11), 2795-2804.
41. Shi, H.; Zhang, Z.; Wang, X.; Li, R.; Hou, W.; Bi, W.; Zhang, X., Inhibition of autophagy induces IL-1p release from ARPE-19 cells via ROS mediated NLRP3 inflammasome activation under high glucose stress. Biochemical and Biophysical Research Communications 2015, 463 (4), 1071-1076.
42. Kannan, R.; Zhang, N.; Sreekumar, P. G.; Spee, C. K.; Rodriguez, A.; Barron, E.; Hinton, D. R., Stimulation of apical and basolateral VEGF-A and VEGF-C secretion by oxidative stress in polarized retinal pigment epithelial cells. Molecular vision 2006, 12, 1649-1659.
43. Atienzar-Aroca, S., Flores-Bellver, M.; Serrano-Heras, G.; Martinez-Gil, N.; Barcia, J. M.; Aparicio, S.; Perez-Cremades, D.; Garcia-Verdugo, J. M.; Diaz-Llopis, M.; Romero, F. J., Oxidative stress in retinal pigment epithelium cells increases exosome secretion and promotes angiogenesis in endothelial cells. Journal of cellular and molecular medicine 2016, 20(8), 1457-1466.
44. DeCicco-Skinner, K. L., Henry, G. H.; Cataisson, C.; Tabib, T., Gwilliam, J. C.; Watson, N. J.; Bullwinkle, E. M.; Falkenburg, L.; O'Neill, R. C.; Morin, A., Endothelial cell tube formation assay for the in vitro study of angiogenesis. JoVE (Journal of Visualized Experiments) 2014, (91), e51312.
45. Li, W.-Q.; Wang, Z.; Hao, S.; He, H.; Wan, Y.; Zhu, C.; Sun, L.-P.; Cheng, G.; Zheng, S.-Y., Mitochondria-targeting polydopamine nanoparticles to deliver doxorubicin for overcoming drug resistance. ACS applied materials & interfaces 2017, 9 (20), 16793-16802.
46. Ball, V., Polydopamine Nanomaterials: Recent Advances in Synthesis Methods and Applications. Front Bioeng Biotechnol 2018, 6, 109-109.
47. Ho, C.-C.; Ding, S.-J., The pH-controlled nanoparticles size of polydopamine for anti-cancer drug delivery. Journal of Materials Science: Materials in Medicine 2013, 24 (10), 2381-2390.
48. Foroozandeh, P.; Aziz, A. A., Insight into Cellular Uptake and Intracellular Trafficking of Nanoparticles. Nanoscale Res Lett 2018, 13 (1), 339-339.
49. Varshochian, R.; Jeddi-Tehrani, M.; Mahmoudi, A. R.; Khoshayand, M. R.; Atyabi, F.; Sabzevari, A.; Esfahani, M. R.; Dinarvand, R. J. E. J. o. P. S., The protective effect of albumin on bevacizumab activity and stability in PLGA nanoparticles intended for retinal and choroidal neovascularization treatments. 2013, 50 (3-4), 341-352.
50. Tiwari, G.; Tiwari, R.; Sriwastawa, B.; Bhati, L.; Pandey, S.; Pandey, P.; Bannerjee, S. K., Drug delivery systems: An updated review. Int. J Pharm Investig 2012, 2 (1), 2-11.
51. Dreyer, D. R.; Miller, D. J.; Freeman, B. D.; Paul, D. R.; Bielawski, C. W., Perspectives on poly (dopamine). Chemical Science 2013, 4 (10), 3796-3802.
52. Ahsan, H.; Ali, A.; Ali, R., Oxygen free radicals and systemic autoimmunity. Clin Exp Immunol 2003, 131 (3), 398-404.
53. Kim, M. H., Chung, J.; Chung, S. M.; Kwag, N. H.; Yoo, J. S., Hydrogen peroxide-induced cell death in a human retinal pigment epithelial cell line, ARPE-19. Korean Journal of Ophthalmology 2003, 17 (1), 19-28.
54. Ballinger, S. W.; Van Houten, B.; Conklin, C. A.; Jin, G.-F.; Godley, B. F., Hydrogen peroxide causes significant mitochondrial DNA damage in human RPE cells. Experimental eye research 1999, 68 (6), 765-772.
55. Pezzella, A.; d'Ischia, M.; Napolitano, A.; Palumbo, A.; Prota, G., An integrated approach to the structure of Sepia melanin. Evidence for a high proportion of degraded 5, 6-dihydroxyindole-2-carboxylic acid units in the pigment backbone. Tetrahedron 1997, 53 (24), 8281-8286.
56. Cave, A. C.; Brewer, A. C.; Narayanapanicker, A.; Ray, R.; Grieve, D. J.; Walker, S.; Shah, A. M., NADPH oxidases in cardiovascular health and disease. Antioxidants & redox signaling 2006, 8 (5-6), 691-728.
57. Jin, L.; Yuan, F.; Chen, C.; Wu, J.; Gong, R.; Yuan, G.; Zeng, H.; Pei, J.; Chen, T., Degradation Products of Polydopamine Restrained Inflammatory Response of LPS-Stimulated Macrophages Through Mediation TLR-4-MYD88 Dependent Signaling Pathways by Antioxidant. Inflammation 2019, 42 (2), 658-671.
58. Kauppinen, A.; Niskanen, H.; Suuronen, T.; Kinnunen, K.; Salminen, A.; Kaarniranta, K., Oxidative stress activates NLRP3 inflammasomes in ARPE-19 cells-Implications for age-related macular degeneration (AMD). Immunology Letters 2012, 147 (1), 29-33.
59. Shen, S.; Wu, Y.; Liu, Y.; Wu, D., High drug-loading nanomedicines: progress, current status, and prospects. International journal of nanomedicine 2017, 12, 4085-4109.
60. Li, F.; Hurley, B.; Liu, Y.; Leonard, B.; Griffith, M., Controlled release of bevacizumab through nanospheres for extended treatment of age-related macular degeneration. Open Ophthalmol J 2012, 6, 54-58.
61. Rose, R. C.; Richer, S. P.; Bode, A. M., Ocular oxidants and antioxidant protection. Proceedings of the Society for Experimental Biology and Medicine 1998, 217 (4), 397-407.
62. Pulido, J. S.; Pulido, C. M.; Bakri, S. J.; McCannel, C. A.; Cameron, J. D., The use of 31-gauge needles and syringes for intraocular injections. Eye 2007, 21 (6), 829-830.
63. Amrite, A. C.; Kompella, U. B., Size-dependent disposition of nanoparticles and microparticles following subconjunctival administration. Journal of pharmacy and pharmacology 2005, 57 (12), 1555-1563.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. An ocular therapeutic composition comprising:
a population of polydopamine (PDA) nanoparticles; wherein each polydopamine nanoparticle is bound to an anti-vascular endothelial growth factor (VEGF) agent.

2. The composition of claim 1, wherein the population of PDA nanoparticles has an average particle size ranging from about 10 nm to about 1000 nm, from about 100 nm to about 200 nm, or from about 120 nm to about 170 nm.

3-5. (canceled)

6. The composition of claim 1, wherein the anti-VEGF agent comprises an antibody or antibody fragment.

7. The composition of claim 1, wherein the anti-VEGF agent comprises bevacizumab, ranibizumab, or aflibercept.

8-9. (canceled)

10. The composition of claim 1, wherein the anti-VEGF agent comprises an inhibitor of a tyrosine kinase stimulated by VEGF.

11. The composition of claim 1, wherein the anti-VEGF agent comprises lapatinib, sunitinib, sorafenib, axitinib, or pazopanib.

12-15. (canceled)

16. The composition of claim 1, wherein each PDA nanoparticle is coated with a polymer.

17. The composition of claim 16, wherein the polymer comprises an alginate.

18. The composition of claim 1, further comprising a hydrogel.

19. The composition of claim 18, wherein the hydrogel comprises an alginate hydrogel or a hyaluronic acid hydrogel.

20. (canceled)

21. The composition of claim 18, wherein the hydrogel comprises poly(N-isopropylacrylamide) grafted sodium hyaluronate hydrogel.

22. A method of treating an ophthalmological disorder in a subject in need thereof comprising injecting into the eye of the subject a therapeutically effective amount of the ocular therapeutic composition of claim 1.

23. The method of claim 22, wherein the ophthalmological disorder is selected from acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, a cancer, and glaucoma.

24. The method of claim 22, wherein the ophthalmological disorder is AMD, neovascularization, macular edema, edema, cataract, glaucoma, diabetic retinopathy, proliferative vitreoretinopathy, posterior capsule opacification, presbyopia, and uveitis.

25. The method of claim 22, wherein injecting into the eye of the subject comprises injecting into the vitreous chamber of the eye.

26. The method of claim 22, wherein injecting into the eye of the subject comprises an intravitreal injection, a subconjunctival injection, a subtenon injection, a retrobulbar injection, or a suprachoroidal injection.

27. A therapeutic composition comprising:
a population of polydopamine (PDA) nanoparticles; wherein each polydopamine nanoparticle is bound to a therapeutic agent;
wherein the therapeutic agent is released upon exposure of the PDA nanoparticle to reactive oxygen species.

28. The composition of claim 27, wherein the population of PDA nanoparticles has an average particle size ranging from about 10 nm to about 1000 nm, from about 100 nm to about 200 nm, or from about 120 nm to about 170 nm.

29-31. (canceled)

32. The composition of claim 27, wherein the therapeutic agent is an anti-vascular endothelial growth factor (VEGF) agent.

33. The composition of claim 27, wherein the anti-VEGF agent is selected from the group consisting of bevacizumab, ranibizumab, aflibercept, lapatinib, sunitinib, sorafenib, axitinib, and pazopanib.
